(19) United States
(12) Patent Application Publication
(10) Pub. No.: US 2025/0121178 A1
(43) Pub. Date: Apr. 17, 2025

(54) Implanted Connector Booster Sealing for Implantable Medical Devices

(71) Applicant: TC1 LLC, St. Paul, MN (US)

(72) Inventors: Daniel I. Harjes, Carlisle, MA (US); Jeff Iudice, Lowell, MA (US); Joseph P. Sylvester, JR., Andover, MA (US); John Hai Nguyen, San Ramon, CA (US); Michael Morado, Escalon, CA (US); Fabian Frigon Franco, Livermore, CA (US); Veera Venkata Jagadeesh Bobba, Alameda, CA (US); Soy Truong, Washington County, OR (US); Chanthy Luy, Tigard, OR (US); Jason Elledge, Portland, OR (US); Lindsay Clough, Portland, OR (US)

(73) Assignee: TC1 LLC, St. Paul, MN (US)

(21) Appl. No.: 18/962,218

(22) Filed: Nov. 27, 2024

Related U.S. Application Data
(63) Continuation of application No. PCT/US2023/023596, filed on May 25, 2023.
(60) Provisional application No. 63/348,416, filed on Jun. 2, 2022.
(60) Provisional application No. 63/391,897, filed on Jul. 25, 2022.

(51) Int. Cl. A61M 60/878; A61M 60/178

(52) U.S. Cl. CPC A61M 60/878; A61M 60/178

(57) ABSTRACT

Described herein is an implantable medical device including a housing, a header coupled to the housing, and a receptacle connector stack disposed in the header. The receptacle connector stack includes a plurality of electrical contacts and a plurality of wiper seals. Each electrical contact of the plurality of electrical contacts is separated by a corresponding wiper seal. A first sealing element is disposed at a proximal end of the receptacle connector stack and a second sealing element is disposed at a distal end of the receptacle connector stack. The sealing elements are mounted on respective seal housings. A third sealing element is disposed adjacent to the first sealing element and further proximal of the connector stack to restrict a bounce back effect caused by one or more of the plurality of wiper seals during insertion of a lead.

### CROSS REFERENCE TO RELATED APPLICATIONS

The present application is a Continuation of PCT/US2023/023596 filed May 25, 2023; which claims the benefit of U.S. Provisional Appln. Nos. 63/348,416 filed Jun. 2, 2022, and 63/391,897 filed Jul. 25, 2022, the disclosures which are incorporated herein by reference in their entirety for all purposes.

### FIELD OF DISCLOSURE

This disclosure relates generally to implantable medical devices and related components. More particularly, this disclosure relates to sealing elements used in the implantable medical devices, such as ventricular assist device controllers.

### BACKGROUND

Ventricular assist devices, known as VADs, are implantable blood pumps used for both short-term (i.e., days, months) and long-term applications (i.e., years or a lifetime) where a patient's heart is incapable of providing adequate circulation, commonly referred to as heart failure or congestive heart failure. According to the American Heart Association, more than five million Americans are living with heart failure, with about 670,000 new cases diagnosed every year. People with heart failure often have shortness of breath and fatigue. Years of living with blocked arteries or high blood pressure can leave a heart too weak to pump enough blood to the body. As symptoms worsen, advanced heart failure develops.

A patient suffering from heart failure, also called congestive heart failure, may use a VAD while awaiting a heart transplant or as a long term destination therapy. In another example, a patient may use a VAD while their own native heart recovers. Thus, a VAD can supplement a weak heart (i.e., partial support) or can effectively replace the natural heart's function. VAD systems of the present invention can be fully implanted in the patient's body and powered by an implantable electrical power source inside the patient's body.

### BRIEF SUMMARY

The present disclosure relates to implantable medical devices, such as a VAD controller, including a connector assembly. The connector assembly includes a female receptacle (referred as a receptacle connector stack) configured to receive a male lead body to establish electrical connection (e.g., power and/or communication) between electronic components disposed in the implantable controller and/or other components of the VAD system (e.g., blood pump, transcutaneous energy transfer system receiver). The connector assembly should be properly sealed to inhibit ingress of fluid inside the connector assembly and associated header from bodily fluids. Also, the connector assembly should provide electrical isolation between contacts. Conventional wiper seals or blades disposed between the contacts typically provide a level of electrical isolation and fluid leakage inhibition while promoting low lead insertion forces. Some implantable medical devices, such as the VAD controller (or high powered devices that have four or more conductors), have rigorous power profiles that require such devices to be on all or most of the time as continuous power is required to operate the VAD system. For example, VAD systems are relatively high powered systems, where the VAD controller supplies average powers of about 3 W to about 6 W, peak powers of about 8 W to about 15 W to operate the VAD. As such, electrical contacts at proximal and distal ends of such high powered medical devices should be additionally isolated to further minimize stray electrical current and fluid leakage from the outside environment for improved device and patient safety, durability, and performance considerations.

The present disclosure provides booster sealing elements disposable in the implantable medical device and configured to inhibit ingress of fluid from a distal end and a proximal end of the receptacle connector stack. For example, the sealing elements may be O-rings. The O-ring can be mounted on an O-ring housing and disposed at the distal end of the receptacle connector stack. Another O-ring may be mounted on another O-ring housing disposed at the proximal end of the receptacle stack. The sealing elements provide an extra layer of contact isolation beyond that of conventional wiper blades. The sealing elements also inhibit any stray electrical currents that may enter or leak to the electrical contacts. Also, the sealing elements may be configured to easily receive the lead without increase in substantial insertion force of the connector assembly. Thus, the sealing elements advantageously provide means to increase the isolation from stray electrical currents between contacts and reduce fluid ingress from the outside environment of the implantable medical device without noticeably increasing the lead insertion force.

It can further be appreciated that an implantable medical device has an overall size small enough to be implanted in a patient. Hence, components within the implantable medical device should be sized to be small enough as well. However, if the sealing elements are scaled too small, manufacturing and assembly of such components can be difficult. For example, during assembly, fingers of a technician may become fatigued over time. The present disclosure provides a seal housing to facilitate proper sizing and ease of assembly of the sealing elements. Further, the sealing elements can be O-rings having an inner diameter in a range from 0.115″ to 0.375″ and outer diameter in a range from 0.145″ to 0.515″ to facilitate ease of assembly and provide improved sealing performance. The O-ring materials that work best are long term implant grade rubber materials with a hardness durometer ranging from 30-90 on the shore A scale. Silicone is a widely used and common implant grade rubber.

During insertion of the lead in the receptacle connector stack, conventional wiper blades in some instances may impart forces on the lead causing the lead to over insert or misalign with contacts. This undesired axial displacement or misalignment of the lead relative to its original insertion location is referred to as a bounce back effect. To address this bounce back effect issue, the present disclosure provides implantable medical devices that further includes a bounce back reducer, such as a sealing element disposed proximal of the receptacle stack. The bounce back reducer advantageously reduces the bounce back effect that can axially displace the lead within the receptacle connector stack for improved alignment between the electrical contacts of the lead and the connector stack. For example, axial shifts in a range from 0.01″ to 0.15″. and can be minimized with the bounce back reducer Also, the bounce back reducer can be retrofitted into other implanted connector designs. The bounce back reducer can be in the form of a canted or cantilevered spring, O-ring, synching feature, or any other complaint member.

Thus, in one aspect, an implantable medical device comprising an implanted connector with booster sealing elements for improved sealing performance is described. The implantable medical device includes a housing, a header coupled to the housing, and at least one receptacle connector stack disposed in the header. The at least one receptacle connector stack includes a plurality of electrical contacts and a plurality of wiper seals. Each electrical contact of the plurality of electrical contacts is separated by a corresponding wiper seal. A first sealing element is disposed at a proximal end of the at least one receptacle connector stack and a second sealing element is disposed at a distal end of the at least one receptacle connector stack.

In many embodiments, the first sealing element is configured to inhibit fluid ingress and stray electrical currents at the proximal end of the at least one receptacle connector stack from an outside environment, and the second sealing element is configured to inhibit fluid ingress and stray electrical currents at the distal end of the at least one receptacle connector stack from the outside environment. The first sealing element and the second sealing element may comprise O-rings. A diameter of each O-rings is within a range of 0.115″ to 0.375″ for the IDs and 0.145″ to 0.515″ for the ODs.

In many embodiments, the implantable medical device further includes a proximal seal housing configured to receive the first sealing element. Likewise, a distal seal housing is configured to receive the second sealing element. In many embodiments, the proximal seal housing and the distal seal housing are separate components assembled with the at least one receptacle connector stack. In some embodiments, the proximal seal housing and the distal seal housing are integrally formed with the at least one receptacle connector stack. The proximal seal housing includes a retention mechanism configured to engage with the at least one receptacle connector stack and the header.

In many embodiments, the retention mechanism includes at least one of a press fit, a crush rib fit, a snap fit, a retaining ring, or a threaded fit. For the press fit, an outer periphery of the proximal seal housing is sized to tightly fit with an inner periphery of the header at a proximal end. For the crush rib fit, the outer periphery of the proximal seal housing includes one or more ribs configured to dig into the inner periphery of the header at the proximal end. For the snap fit, the outer periphery of the proximal seal housing includes one or more snapping elements configured to engage with the inner periphery of the header at the proximal end. For the retaining ring, the retaining ring is attached on the outer periphery of the proximal seal housing to hold the proximal housing in place against the housing and restrict axial movement at the proximal end. For the threaded fit, the outer periphery of the proximal seal housing includes threads configured to engage with threads on the inner periphery of the housing at the proximal end.

In many embodiments, the implantable medical device further includes a third sealing element disposed adjacent to the first sealing element and further proximal of the at least one receptacle connector stack to restrict a bounce back effect caused by one or more of the plurality of wiper seals. In many embodiments, the implantable medical device is a ventricular assist device (VAD) implantable controller configured to generate control signals to control a blood flow and provide power to the VAD. In many embodiments, the header includes two receptacle connectors stacks spaced apart from each other, each connector stack including six contacts for power and communication with the VAD and TETS. The at least one receptacle connector stack includes a first receptacle connector stack spaced from and electrically isolated from a second receptacle connector stack.

In another aspect, a fully implantable left ventricle assist system (FILVAS) includes a heart pump, an implantable transcutaneous energy transmission system (TETS) receiver, a first implantable lead, a second implantable lead, and an implantable controller. The heart pump is configured for pumping blood from a ventricle of a heart of a patient to an artery to supplement or replace pumping of blood by the ventricle to the artery. The TETS receiver is configured for receiving and transmitting power to continuously operate the heart pump. The implantable controller is communicably coupled to the heart pump via the first implantable lead and to the TETS receiver via the second implantable lead.

In many embodiments, the implantable controller includes a housing, a header coupled to the housing, a first receptacle connector stack disposed in the header and configured to receive the first implantable lead and establish an electrical coupling between the implantable controller and the heart pump, and a second receptacle connector stack disposed in the header and configured to receive the second implantable lead and establish an electrical coupling between the implantable controller and the TETS receiver. Each of the first receptacle connector stack and the second receptacle connector stack includes a plurality of electrical contacts configured to couple with the respective implantable lead, a plurality of wiper seals, each electrical contact being isolated by a corresponding wiper seal, a proximal sealing element disposed at a proximal end of the respective receptacle connector stack, and a distal sealing element disposed at a distal end of the respective receptacle connector stack. In many embodiments, the proximal sealing elements and the distal sealing elements are O-rings.

In many embodiments, the fully implantable left ventricle assist system further includes, for each of the first receptacle connector stack and the second receptacle connector stack, a proximal seal housing configured to receive the proximal sealing element and coupleable to the proximal end of the respective receptacle connector stack. A distal seal housing is configured to receive the distal sealing element and is coupleable to the distal end of the respective receptacle connector stack. Each proximal seal housing includes a retention mechanism configured to engage with the respective receptacle connector stack and the header.

In many embodiments, the header further includes a bounce back reducer disposed within the header and located proximal of the respective proximal sealing element. The bounce back reducer is configured to engage the respective implantable lead and restrict a bounce back effect experienced by the respective implantable lead that are caused by forces exerted by one or more of the plurality of wiper seals upon insertion of the respective implantable lead into the respective receptacle connector stack. In many embodiments, the bounce back reducer is a canted spring, synching feature, an O-ring, or a complaint member. In many embodiments, the header further includes a cassette with a groove to receive the bounce back reducer.

In many embodiments, the header further includes a vent or a septum located distal to the distal sealing element of the respective receptacle connector stack to prevent hydrostatic locking between the respective implantable lead and the respective receptacle connector stack. The distal sealing element inhibits ingress of fluid from the vent or the septum. In many embodiments, the first implantable lead includes electrical contacts configured to couple with a corresponding electrical contacts of the first receptacle connector stack, and an electrical cable to facilitate communication and power between the implantable controller and the heart pump. The second implantable lead comprises electrical contacts configured to couple with a corresponding electrical contact of the second receptacle connector stack, and an electrical cable to facilitate communication and power between the implantable controller and the TETS receiver. The first receptacle connector stack is spaced from and electrically isolated from the second receptacle connector stack by the respective proximal sealing elements and the respective distal sealing elements.

In another aspect, an implantable lead of an implantable medical device is described. The implantable lead includes an elongate shaft, a stem, a plurality of electrical contacts, a strength member, and a plurality of wires. The elongate shaft has a proximal end and a distal end, the elongate shaft having a diameter of approximately 3.2 mm, a stem coupled to the proximal end of the elongate shaft. The plurality of electrical contacts are disposed on an outer surface of the elongate shaft and axially spaced from each other. The plurality of electrical contacts include first and second electrical contacts for power transmission, third and fourth electrical contacts for communication, and fifth and sixth electrical contacts serving as ground. The strength member is disposed axially within the elongate shaft. The plurality of wires are electrically coupled to the plurality of electrical contacts and extending axially within the elongate shaft. The plurality of wires include a first wire and a second wire electrically coupled to the first and the second electrical contacts, respectively; a third wire and a fourth wire electrically coupled to the third and the fourth electrical contacts, respectively; and a fifth wire and a sixth wire electrically coupled to the fifth and the sixth electrical contacts, respectively. The strength member and the plurality of wires are electrically isolated from each other are configured to be constrained within the diameter of the elongate shaft.

In many embodiments, the plurality of wires are radially disposed approximately 60 degree from each other and circumferentially around the strength member. In many embodiments, each of the plurality of electrical contacts comprises a ring contact, wherein the ring contacts are coaxial and linearly spaced from each other along the elongate shaft. In many embodiments, the electrical contacts and the electrical wires are made of corrosion resistant and electrically conductive material. In many embodiments, the electrical contacts and the wires are made of platinum iridium or a nickel-cobalt base alloy of a multiphase alloy. In many embodiments, each of the plurality of wires have a diameter of approximately 0.0015 mm and a varying exposed length from the stem in a range from 7 mm to 8 mm and the strength member has a diameter in a range from 0.5 to 1.5 mm and a length in a range for 28 mm to 30 mm.

In many embodiments, the implantable lead further includes an insulation cover disposed over each wire of the plurality of wires. In many embodiments, the plurality of electrical contacts are separated by a molded insulative material for electrical isolation. In many embodiments, the first and the second electrical contacts for power transmission are disposed at a distal portion of the elongate shaft. The fifth and the sixth electrical contacts serving as ground are disposed at a proximal portion of the elongate shaft. The third and the fourth electrical contacts for communication are disposed between the proximal portion and the distal portion of the elongate shaft.

In many embodiments, the stem includes a shoulder at a distal portion of the stem where the elongate shaft is configured to receive a sealing element of a header assembly, and a groove configured to receive a locking assembly for securely coupling the lead to the header assembly of a second implantable medical device. In many embodiments, the implantable lead comprises a ventricular assist device (VAD) lead or a transcutaneous energy transmission system (TETS) receiver lead. The implantable lead is coupled to a receptacle connector stack of an implantable controller.

In another aspect, a fully implantable left ventricle assist system (FILVAS) is described. The system includes an implantable heart pump, an implantable transcutaneous energy transmission system (TETS) receiver, a first implantable lead, a second implantable lead, and an implantable controller. The implantable heart pump configured for pumping blood from a ventricle of a heart of a patient to an artery to supplement or replace pumping of blood by the ventricle to the artery. The implantable transcutaneous energy transmission system (TETS) receiver configured for receiving and transmitting power to continuously operate the implantable heart pump. The first implantable lead coupled to the implantable heart pump. The first lead includes a first elongate shaft, a first set of six electrical contacts coaxially mounted on an outer surface of the first elongate shaft, a first set of six wires disposed within the first elongate shaft, each wire electrically coupled to a respective electrical contact of the first set of six electrical contacts at one end and to the implantable heart pump at an opposite end; and a first strength member disposed within the first elongate shaft and electrically isolated from the first set of six wires. The second implantable lead coupled to the TETS receiver. The second lead includes a second elongate shaft, a second set of six electrical contacts coaxially mounted on an outer surface of the second elongate shaft, a second set of six wires disposed within the second elongate shaft, each wire electrically coupled to a respective electrical contact of the second set of six electrical contacts at one end and the TETS receiver at an opposite end; and a second strength member disposed within the second elongate shaft and electrically isolated from the second set of six wires. The second implantable lead is larger in diameter than the first implantable lead. The implantable controller includes a first receptacle connector stack configured to receive the first implantable lead to electrically couple the implantable heart pump to the implantable controller, and a second receptacle connector stack configured to receive the second implantable lead to electrically couple the TETS receiver to the implantable controller. The first receptacle connector stack spaced apart from the second receptacle connector stack.

In many embodiments, the first set of six electrical contacts of the first implantable lead comprises a first and a second electrical contacts for power transmission, a third and a fourth electrical contacts for communication, and a fifth and a sixth electrical contacts for serving as ground. The second set of six electrical contacts of the second implantable lead comprises a first and a second electrical contacts for power transmission, a third and a fourth electrical contacts for communication, and a fifth and a sixth electrical contacts for serving as ground. In many embodiments, the first implantable lead includes the first and the second electrical contacts for power transmission are disposed at a distal portion of the first implantable lead. The fifth and the sixth electrical contacts serving as ground are disposed at a proximal portion of the first implantable lead. The third and the fourth electrical contacts for communication are disposed between the proximal portion and the distal portion of the first implantable lead.

In many embodiments, the second implantable lead includes a first electrical contact serving as a first volt supply disposed at a distal portion of the second implantable lead, a second electrical contact serving as a ground disposed adjacent and proximal the first electrical contact, a third electrical contact serving as a power terminal disposed adjacent and proximal the second electrical contact, a fourth electrical contact serving as a communication line disposed adjacent and proximal the third electrical contact, a fifth electrical contact serving as another ground disposed adjacent to and proximal the fourth electrical contact, and a sixth electrical contact serving as another power terminal disposed at a proximal portion of the second implantable lead.

In many embodiments, a proximal portion of the second implantable lead is larger in diameter than a proximal portion of the first implantable lead. In many embodiments, the first implantable lead includes a first stem disposed at a proximal portion of the first elongate shaft and the second implantable lead includes a second stem disposed at a proximal portion of the second elongate shaft. A diameter of the second stem is larger than a diameter of the first stem. Each of the first stem and the second stem includes a shoulder for mounting a seal and a groove for locking the respective lead to a header assembly of the implantable controller. In many embodiments, the first receptacle connector stack and the second receptacle connector stack each include a proximal sealing element and a distal sealing element. In many embodiments, each of the first implantable lead and the second implantable lead do not require tightening a set screw to electrically activate one or more lines of conductions.

In another aspect, an implantable lead of an implantable medical device is described. The lead includes a stepped diameter elongate shaft, a plurality of electrical contacts, and a plurality of wires. The stepped diameter elongate shaft having a proximal portion and a distal portion, the proximal portion being larger in diameter than the distal portion. The plurality of electrical contacts disposed on an outer surface of the stepped diameter elongate shaft and axially spaced from each other, the plurality of electrical contacts comprising first and second electrical contacts for power transmission, third and fourth electrical contacts for communication, and fifth and sixth electrical contacts serving as ground, wherein one or more of the electrical contacts have different diameters. The plurality of wires electrically coupled to the plurality of electrical contacts and extending axially within the stepped diameter elongate shaft. The plurality of wires comprise a first wire and a second wire electrically coupled to the first and the second electrical contacts, respectively; a third wire and a fourth wire electrically coupled to the third and the fourth electrical contacts, respectively; and a fifth wire and a sixth wire electrically coupled to the fifth and the sixth electrical contacts, respectively.

In many embodiments, the stepped diameter elongate shaft includes a first diameter portion configured to receive the first and the second electrical contacts for power transmission at the distal portion of the elongate shaft; a second diameter portion configured to receive the third and the fourth electrical contacts for communication between the distal portion and the proximal portion of the elongate shaft; and a third diameter portion configured to receive the fifth and the sixth electrical contacts serving as ground at the proximal portion of the elongate shaft. The second diameter portion has a larger diameter than the first diameter portion, and the third diameter portion has a larger diameter than the second diameter portion. In many embodiments, the stepped diameter elongate shaft includes a tapered diameter increasing from the distal portion to the proximal portion, wherein each electrical contact of the six electrical contacts has a different diameter. Optionally, the implantable lead further comprises a tapered strength member disposed axially within the stepped diameter.

The forgoing general description of the illustrative implementations and the following detailed description thereof are merely exemplary aspects of the teachings of this disclosure, and are not restrictive.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and can or cannot represent actual or preferred values or dimensions.

FIG. 1 illustrates a medical system that includes an implantable transcutaneous energy transmission system receiver, an implantable controller, and a ventricular assist device (VAD), in accordance with many embodiments.

FIG. 2 is an illustration of a blood circulatory support system that includes the VAD implanted in a patient's body, in accordance with many embodiments.

FIG. 3 is an exploded view of implanted components of the circulatory support system of FIG. 2.

FIG. 4 is an illustration of the VAD of FIG. 1 attached to the patient's heart to augment blood pumping by the patient's left ventricle.

FIG. 5 is a cross-sectional view of the VAD of FIG. 4.

FIG. 6 is a schematic diagram of an embodiment of the implanted controller of FIG. 1.

FIG. 7 is a cross-sectional view of a portion of a header and a receptacle connector stack with sealing elements disposed therein of the implantable controller of FIG. 1.

FIG. 8 illustrates the header with the female receptacle connector stack disposed therein.

FIG. 9 illustrates a male lead configured to be received in the receptacle connector stack of FIG. 8

FIG. 10 illustrates an assembly of the lead within the receptacle connector stack of FIG. 8 and FIG. 9.

FIG. 11A illustrates a snapping means for attaching the receptacle connector stack to the header, in accordance with many embodiments.

FIG. 11B shown an enlarged portion a snapping element of the snapping means of FIG. 11A.

FIG. 12A illustrates a threaded attachment means for coupling the receptacle connector stack to the header, in accordance with many embodiments.

FIG. 12B illustrates the receptacle connector stack inserted in the header and coupled via the threaded means of FIG. 12A.

FIG. 13A illustrates a retaining ring used for coupling the receptacle connector stack to the header, in accordance with many embodiments.

FIG. 13B illustrates an enlarged portion with retaining ring of FIG. 13A coupled to the receptacle connector stack.

FIG. 14A illustrates a press fit used for coupling the receptacle connector stack to the header, in accordance with many embodiments.

FIG. 14B illustrates a cross-section of a press fitted receptacle connector stack of FIG. 14A.

FIG. 15A illustrates a crush rib fit used for coupling the receptacle connector stack to the header, in accordance with many embodiments.

FIG. 15B illustrates a cross-section of a crush rib fitted receptacle connector stack of FIG. 15A.

FIG. 16 illustrates a perspective view of an implantable lead, in accordance with many embodiments.

FIG. 17 illustrates a front view of an implantable lead couplable to an implantable medical device (e.g., ventricular assist device), in accordance with many embodiments.

FIG. 18 illustrates a front view of another implantable lead couplable to another implantable medical device (e.g., TETS receiver), in accordance with many embodiments.

FIG. 19 illustrates a cross-section of any of the implantable leads of FIGS. 16-18.

FIG. 20 illustrates a cross-section of a stem of any of the implantable leads of FIGS. 16-18.

FIG. 21 illustrates an implantable controller coupled with a header, in accordance with many embodiments.

FIG. 22 illustrates a stepped diameter implantable lead and a corresponding receptacle connector stack, according to another embodiment.

FIG. 23 is a perspective view of elongated shaft of the stepped diameter implantable lead of FIG. 22.

FIG. 24 is a front cross-section view of the stepped diameter implantable lead of FIG. 22

FIG. 25 illustrates an implantable lead of an implantable medical device with four electrical contacts, in accordance with another embodiment.

FIG. 26 illustrates another implantable lead of another implantable medical device with four electrical contacts, in accordance with another embodiment.

FIG. 27 illustrates an implantable lead with three electrical contacts, in accordance with another embodiment.

### DETAILED DESCRIPTION

The description set forth below in connection with the appended drawings is intended as a description of various embodiments of the disclosed subject matter and is not necessarily intended to represent the only embodiment(s). In certain instances, the description includes specific details for the purpose of providing an understanding of the disclosed embodiment(s). However, it will be apparent to those skilled in the art that the disclosed embodiment(s) can be practiced without those specific details. In some instances, well-known structures and components can be shown in block diagram form in order to avoid obscuring the concepts of the disclosed subject matter. In the drawings, like reference numerals represent like parts throughout the several views

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics can be combined in any suitable manner in one or more embodiments. Further, it is intended that embodiments of the disclosed subject matter cover modifications and variations thereof.

It is to be understood that terms such as “distal,” “proximal,” “top,” “front,” “side,” “inner,” and the like that can be used herein merely describe points of reference and do not necessarily limit embodiments of the present disclosure to any particular orientation or configuration. As used herein, “proximal” refers to a direction toward the end of the receptacle connector stack near the clinician and “distal” refers to a direction away from the clinician and (generally) inside the body of a patient. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

The terms “longitudinal,” “axial” or “axially” are generally longitudinal as used herein to describe the relative position related to a receptacle connector stack or other components of the system herein. For example, “longitudinal” or “axial” indicates an axis passing along a center of a receptacle connector stack from a proximal end to a distal end. The term “radial” generally refers to a direction perpendicular to the “axial” direction.

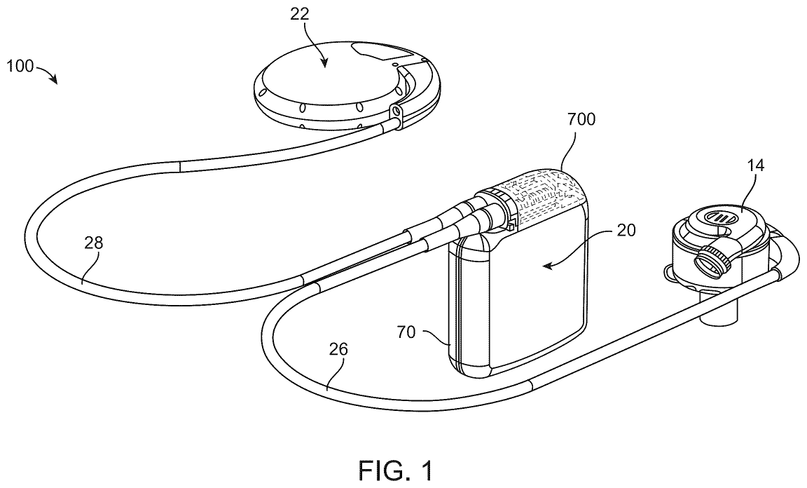

FIG. 1 illustrates a medical system 100 including an implantable medical device 20 coupleable to one or more components of the medical system used for treating a medical condition of a patient. The implantable medical device 20 can be a controller configured to control and/or provide power to the one or more components of the medical system. Accordingly, the implantable medical device 20 may also be referred as an implantable controller 20. The implantable controller 20 includes a housing 70 and a header 700 coupled to the housing 70. In many embodiments, the housing 70 can be made of titanium or like biocompatible materials and the header 700 can be made of a resin (e.g., epoxy or other polymers) or plastic by molding it on the housing 70. The housing 70 is hermetically sealed.

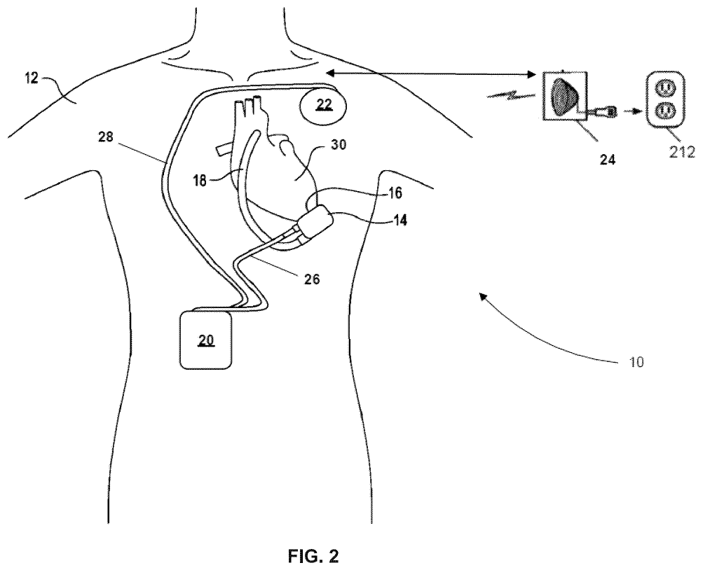

FIG. 2 illustrates a blood circulation assist system 10 using the implantable controller 20 in conjunction with a ventricular assist device (VAD) 14 implanted in a patient 12. In many embodiments described herein, the blood circulatory assist system 10 includes the implantable VAD 14 coupled to a patient's heart 30, the implantable controller 20 that controls operation of the VAD 14 and powers the VAD 14, and an implantable transcutaneous energy transfer system (TETS) power receiver 22 that supplies power to the implantable controller 20. The VAD 14 as illustrated can be a centrifugal flow blood pump, or an axial flow blood pump, that can be operated in a pulsatile mode that produces a blood pressure pulse via an increase in the rotational speed of the VAD or in a constant speed mode in which the VAD rotational rate is held constant.

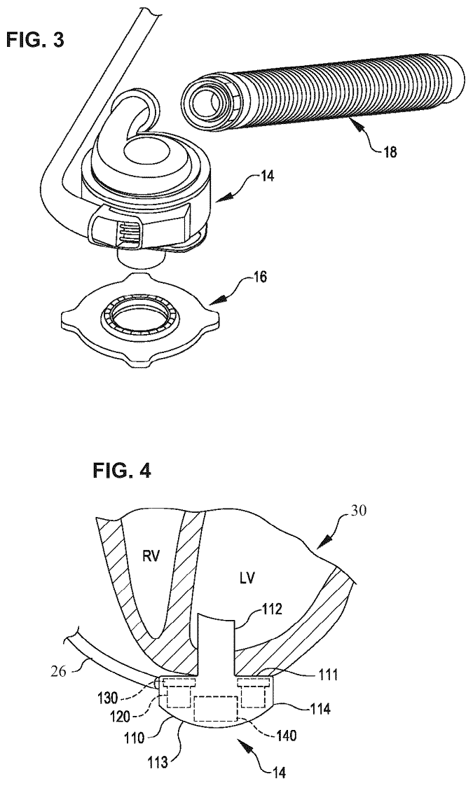

Referring to FIGS. 1 through 3, the blood circulation assist system 10 further includes an implantable ventricular cuff 16, an implantable outflow cannula 18, an external TETS power transmitter 24, an implantable controller-to-VAD connection cable 26, and an implantable TETS receiver-to-controller connection cable 28. The VAD 14 can be coupled to an apex of the left ventricle via the ventricular cuff 16, as illustrated, or the right ventricle, or a separate VAD can be attached to each of the ventricles of the heart 30. Accordingly, depending on the medical application, the system 10 can also be referred as a fully implantable left ventricular assist system (FILVAS) 10. Examples of such FILVAS systems are further described in U.S. Pat. Nos. 8,562,508 and 9,079,043, all of which are incorporated herein by reference for all purposes in their entirety.

The VAD 14, as described in more detail below, can be capable of pumping the entire flow of blood delivered to the left ventricle from the pulmonary circulation (i.e., up to 10 liters per minute). Related blood pumps applicable to the present disclosure are also described in in U.S. Pat. Nos. 5,695,471, 6,071,093, 6,116,862, 6,186,665, 6,234,772, 6,264,635, 6,688,861, 7,699,586, 7,976,271, 7,997,854, 8,007,254, 8,152,493, 8,419,609, 8,652,024, 8,668,473, 8,852,072, 8,864,643, 8,882,744, 9,068,572, 9,091,271, 9,265,870, and 9,382,908, all of which are incorporated herein by reference for all purposes in their entirety. The VAD 14 can be attached to the heart 30 via the ventricular cuff 16, which can be sewn to the heart 30 and coupled to the VAD 14. In the illustrated embodiment, the output of the VAD 14 connects to the ascending aorta via the outflow cannula 18 so that the VAD 14 effectively diverts blood from the left ventricle and propels it to the aorta for circulation through the rest of the patient's vascular system.

The controller-to-VAD connection cable 26 connects the VAD 14 to the implantable controller 20, which monitors the system 10 operation. Related controller systems applicable to the present disclosure are described in greater detail below and in U.S. Pat. Nos. 5,888,242, 6,991,595, 8,323, 174, 8,449,444, 8,506,471, 8,597,350, and 8,657,733, EP 1812094, and U.S. Patent Publication Nos. 2005/0071001 and 2013/0314047, all of which are incorporated herein by reference for all purposes in their entirety. The implantable controller 20 is configured to supply power to and control operation of the VAD 14. The implantable controller 20 is configured to be implanted within the patient 12 in a suitable location spaced apart from the VAD 14 and operatively coupled with the VAD 14 via the controller-to-VAD connection cable 26.

The TETS power receiver 22 is configured to wirelessly receive power transmitted by the external TETS power transmitter 24, which is outside the body for powering operation of the system 10. The TETS power receiver 22 is configured to be implanted within the patient 12 in a suitable location spaced apart from the VAD 14 and the controller 20. The TETS power receiver 22 is operatively coupled with and supplies power to the controller 20 via the implantable TETS power receiver-to-controller connection cable 28. Further, the TETS power transmitter 24 is configured to be coupled to an electric power source 212 such as an electrical wall outlet or other suitable external power sources.

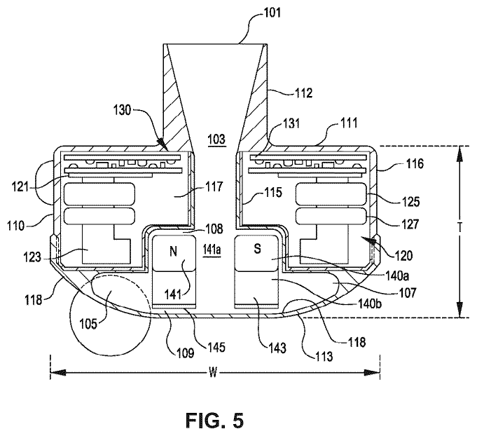

With reference to FIG. 4 and FIG. 5, the VAD 14 has a circular or puck-shaped housing 110 and is shown implanted within the patient 12 with a first face 111 of the housing 110 positioned against the patient's heart 30 and a second face 113 of the housing 110 facing away from the heart 30. The first face 111 of the housing 110 includes an inlet cannula 112 extending into the left ventricle LV of the heart 30. The second face 113 of the housing 110 has a chamfered edge 114 to avoid irritating other tissue that may come into contact with the VAD 14, such as the patient's diaphragm. To construct the illustrated shape of the puck-shaped housing 110 in a compact form, a stator 120 and electronics 130 of the VAD 14 are positioned on the inflow side of the housing toward first face 111, and a rotor 140 of the VAD 14 is positioned along the second face 113. This positioning of the stator 120, electronics 130, and rotor 140 permits the edge 114 to be chamfered along the contour of the rotor 140, as illustrated in at least FIG. 4 and FIG. 5, for example.

Referring to FIG. 5, the VAD 14 includes a dividing wall 115 within the housing 110 defining a blood flow conduit 103. The blood flow conduit 103 extends from an inlet opening 101 of the inlet cannula 112 through the stator 120 to an outlet opening 105 defined by the housing 110. The rotor 140 is positioned within the blood flow conduit 103. The stator 120 is disposed circumferentially about a first portion 140a of the rotor 140, for example about a permanent magnet 141. The stator 120 is also positioned relative to the rotor 140 such that, in use, blood flows within the blood flow conduit 103 through the stator 120 before reaching the rotor 140. The permanent magnet 141 has a permanent magnetic north pole N and a permanent magnetic south pole S for combined active and passive magnetic levitation of the rotor 140 and for rotation of the rotor 140. The rotor 140 also has a second portion 140b that includes impeller blades 143. The impeller blades 143 are located within a volute 107 of the blood flow conduit such that the impeller blades 143 are located proximate to the second face 113 of the housing 110.

The puck-shaped housing 110 further includes a peripheral wall 116 that extends between the first face 111 and a removable cap 118. As illustrated, the peripheral wall 116 is formed as a hollow circular cylinder having a width W between opposing portions of the peripheral wall 116. The housing 110 also has a thickness T between the first face 111 and the second face 113 that is less than the width W. The thickness T is from about 0.5 inches to about 1.5 inches, and the width W is from about 1 inch to about 4 inches. For example, the width W can be approximately 2 inches, and the thickness T can be approximately 1 inch.

The peripheral wall 116 encloses an internal compartment 117 that surrounds the dividing wall 115 and the blood flow conduit 103, with the stator 120 and the electronics 130 disposed in the internal compartment 117 about the dividing wall 115. The removable cap 118 includes the second face 113, the chamfered edge 114, and defines the outlet opening 105. The cap 118 can be threadedly engaged with the peripheral wall 116 to seal the cap 118 in engagement with the peripheral wall 116. The cap 118 includes an inner surface 118a of the cap 118 that defines the volute 107 that is in fluid communication with the outlet opening 105.

Within the internal compartment 117, the electronics 130 are positioned adjacent to the first face 111 and the stator 120 is positioned adjacent to the electronics 130 on an opposite side of the electronics 130 from the first face 111. The electronics 130 include circuit boards 131 and various components carried on the circuit boards 131 to control the operation of the VAD 14 (e.g., magnetic levitation and/or drive of the rotor) by controlling the electrical supply to the stator 120. The housing 110 is configured to receive the circuit boards 131 within the internal compartment 117 generally parallel to the first face 111 for efficient use of the space within the internal compartment 117. The circuit boards also extend radially-inward towards the dividing wall 115 and radially-outward towards the peripheral wall 116. For example, the internal compartment 117 is generally sized no larger than necessary to accommodate the circuit boards 131, and space for heat dissipation, material expansion, potting materials, and/or other elements used in installing the circuit boards 131. Thus, the external shape of the housing 110 proximate the first face 111 generally fits the shape of the circuits boards 131 closely to provide external dimensions that are not much greater than the dimensions of the circuit boards 131.

With continued reference to FIG. 5, the stator 120 includes a back iron 121 and pole pieces 123a-123f arranged at intervals around the dividing wall 115. The back iron 121 extends around the dividing wall 115 and is formed as a generally flat disc of a ferromagnetic material, such as steel, in order to conduct magnetic flux. The back iron 121 is arranged beside the control electronics 130 and provides a base for the pole pieces 123a-123f.

Each of the pole piece 123a-123f is L-shaped and has a drive coil 125 for generating an electromagnetic field to rotate the rotor 140. For example, the pole piece 123a has a first leg 124a that contacts the back iron 121 and extends from the back iron 121 towards the second face 113. The pole piece 123a can also have a second leg 124b that extends from the first leg 124a through an opening of a circuit board 131 towards the dividing wall 115 proximate the location of the permanent magnet 141 of the rotor 140. In an aspect, each of the second legs 124b of the pole pieces 123a-123f is sticking through an opening of the circuit board 131. In an aspect, each of the first legs 124a of the pole pieces 123a-123f is sticking through an opening of the circuit board 131. In an aspect, the openings of the circuit board are enclosing the first legs 124a of the pole pieces 123a-123f.

In a general aspect, the VAD 14 can include one or more Hall sensors that may provide an output voltage, which is directly proportional to a strength of a magnetic field that is located in between at least one of the pole pieces 123a-123f and the permanent magnet 141, and the output voltage may provide feedback to the control electronics 130 of the VAD 14 to determine if the rotor 140 and/or the permanent magnet 141 is not at its intended position for the operation of the VAD 14. For example, a position of the rotor 140 and/or the permanent magnet 141 can be adjusted, e.g., the rotor 140 or the permanent magnet 141 may be pushed or pulled towards a center of the blood flow conduit 103 or towards a center of the stator 120.

Each of the pole pieces 123a-123f also has a levitation coil 127 for generating an electromagnetic field to control the radial position of the rotor 140. Each of the drive coils 125 and the levitation coils 127 includes multiple windings of a conductor around the pole pieces 123a-123f. Particularly, each of the drive coils 125 is wound around two adjacent ones of the pole pieces 123, such as pole pieces 123d and 123e, and each levitation coil 127 is wound around a single pole piece. The drive coils 125 and the levitation coils 127 are wound around the first legs of the pole pieces 123, and magnetic flux generated by passing electrical current though the coils 125 and 127 during use is conducted through the first legs and the second legs of the pole pieces 123 and the back iron 121. The drive coils 125 and the levitation coils 127 of the stator 120 are arranged in opposing pairs and are controlled to drive the rotor and to radially levitate the rotor 140 by generating electromagnetic fields that interact with the permanent magnetic poles S and N of the permanent magnet 141. Because the stator 120 includes both the drive coils 125 and the levitation coils 127, only a single stator is needed to levitate the rotor 140 using only passive and active magnetic forces. The permanent magnet 141 in this configuration has only one magnetic moment and is formed from a monolithic permanent magnetic body 141. For example, the stator 120 can be controlled as discussed in U.S. Pat. No. 6,351,048, the entire contents of which are incorporated herein by reference for all purposes. The control electronics 130 and the stator 120 receive electrical power, data, and control signals from the implanted controller 20 via the controller-to-VAD connection cable 26 (FIG. 4). Further related patents, namely U.S. Pat. Nos. 5,708,346, 6,053,705, 6,100,618, 6,222,290, 6,249,067, 6,278,251, 6,351,048, 6,355,998, 6,634,224, 6,879,074, and 7,112,903, are incorporated herein by reference for all purposes in their entirety.

The rotor 140 is arranged within the housing 110 such that its permanent magnet 141 is located upstream of impeller blades in a location closer to the inlet opening 101. The permanent magnet 141 is received within the blood flow conduit 103 proximate the second legs 124b of the pole pieces 123 to provide the passive axial centering force though interaction of the permanent magnet 141 and ferromagnetic material of the pole pieces 123. The permanent magnet 141 of the rotor 140 and the dividing wall 115 form a gap 108 between the permanent magnet 141 and the dividing wall 115 when the rotor 140 is centered within the dividing wall 115. The gap 108 may be from about 0.2 millimeters to about 2 millimeters. For example, the gap 108 can be approximately 1 millimeter. The north permanent magnetic pole N and the south permanent magnetic pole S of the permanent magnet 141 provide a permanent magnetic attractive force between the rotor 140 and the stator 120 that acts as a passive axial centering force that tends to maintain the rotor 140 generally centered within the stator 120 and tends to resist the rotor 140 from moving towards the first face 111 or towards the second face 113. When the gap 108 is smaller, the magnetic attractive force between the permanent magnet 141 and the stator 120 is greater, and the gap 108 is sized to allow the permanent magnet 141 to provide the passive magnetic axial centering force having a magnitude that is adequate to limit the rotor 140 from contacting the dividing wall 115 or the inner surface 118a of the cap 118. The rotor 140 also includes a shroud 145 that covers the ends of the impeller blades 143 facing the second face 113 that assists in directing blood flow into the volute 107. The shroud 145 and the inner surface 118a of the cap 118 form a gap 109 between the shroud 145 and the inner surface 118a when the rotor 140 is levitated by the stator 120. The gap 109 is from about 0.2 millimeters to about 2 millimeters. For example, the gap 109 is approximately 1 millimeter.

As blood flows through the blood flow conduit 103, blood flows through a central aperture 141a formed through the permanent magnet 141. Blood also flows through the gap 108 between the rotor 140 and the dividing wall 115 and through the gap 109 between the shroud 145 and the inner surface 108a of the cap 118. The gaps 108 and 109 are large enough to allow adequate blood flow to limit clot formation that may occur if the blood is allowed to become stagnant. The gaps 108 and 109 are also large enough to limit pressure forces on the blood cells such that the blood is not damaged when flowing through the VAD 14. As a result of the size of the gaps 108 and 109 limiting pressure forces on the blood cells, the gaps 108 and 109 are too large to provide a meaningful hydrodynamic suspension effect. That is to say, the blood does not act as a bearing within the gaps 108 and 109, and the rotor is only magnetically-levitated. In various embodiments, the gaps 108 and 109 are sized and dimensioned so the blood flowing through the gaps forms a film that provides a hydrodynamic suspension effect. In this manner, the rotor can be suspended by magnetic forces, hydrodynamic forces, or both.

Because the rotor 140 is radially suspended by active control of the levitation coils 127 as discussed above, and because the rotor 140 is axially suspended by passive interaction of the permanent magnet 141 and the stator 120, no magnetic-field generating rotor levitation components are needed proximate the second face 113. The incorporation of all the components for rotor levitation in the stator 120 (i.e., the levitation coils 127 and the pole pieces 123) allows the cap 118 to be contoured to the shape of the impeller blades 143 and the volute 107. Additionally, incorporation of all the rotor levitation components in the stator 120 eliminates the need for electrical connectors extending from the compartment 117 to the cap 118, which allows the cap to be easily installed and/or removed and eliminates potential sources of pump failure.

In use, the drive coils 125 of the stator 120 generates electromagnetic fields through the pole pieces 123 that selectively attract and repel the magnetic north pole N and the magnetic south pole S of the rotor 140 to cause the rotor 140 to rotate within stator 120. For example, the one or more Hall sensors may sense a current position of the rotor 140 and/or the permanent magnet 141, wherein the output voltage of the one or more Hall sensors may be used to selectively attract and repel the magnetic north pole N and the magnetic south pole S of the rotor 140 to cause the rotor 140 to rotate within stator 120. As the rotor 140 rotates, the impeller blades 143 force blood into the volute 107 such that blood is forced out of the outlet opening 105. Additionally, the rotor draws blood into VAD 14 through the inlet opening 101. As blood is drawn into the blood pump by rotation of the impeller blades 143 of the rotor 140, the blood flows through the inlet opening 101 and flows through the control electronics 130 and the stator 120 toward the rotor 140. Blood flows through the aperture 141a of the permanent magnet 141 and between the impeller blades 143, the shroud 145, and the permanent magnet 141, and into the volute 107. Blood also flows around the rotor 140, through the gap 108 and through the gap 109 between the shroud 145 and the inner surface 118a of the cap 118. The blood exits the volute 107 through the outlet opening 105, which may be coupled to the outflow cannula 18.

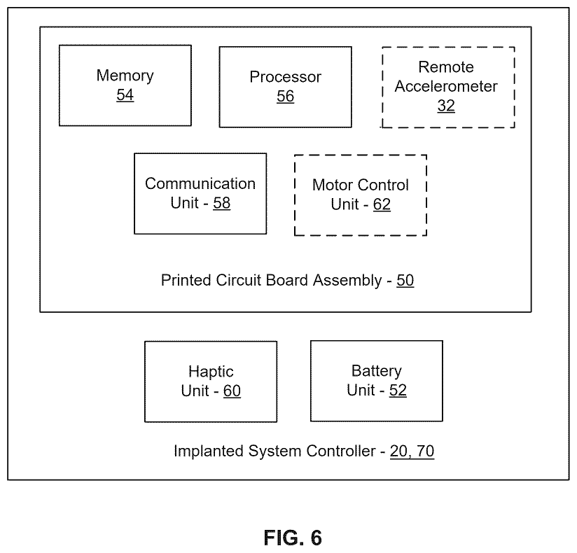

FIG. 6 is a schematic diagram of the embodiment of the implantable controller 20 of FIG. 1. The implantable controller 20 includes a printed circuit board assembly (PCBA) 50, a controller battery unit 52, and a haptic unit 60. In the illustrated embodiment, the PCBA 50 includes a memory 54, a processor 56, and a communication unit 58. In some embodiments, the PCBA 50 includes a motor control unit 62 configured to control drive currents supplied to the motor stator 120 of the VAD 14 to control the rotational rate of the rotor/impeller 140 to control the flow rate of the blood flow through the VAD 14. In some embodiments, PCBA 50 can optionally include a remote accelerometer 32 to measure accelerations induced via operation of the VAD 14. The remote accelerometer 32 can alternately be attached to the inner surface of the housing 70 and operatively coupled with another PCBA.

The memory 54 can store suitable instructions executable by the processor 56 for processing, for example, automatically adjust VAD impeller rotational speed in response to the physiologic demand of the patient, determine any suitable number of physiologic states of the patient, monitor conditions of the VAD 14, and/or other functionalities associated with other components such as the remote accelerometer. The controller battery unit 52 can store energy used to operate the VAD 14, the controller 20, and/or the TETS power receiver 22 during time periods when power is not being received by the TETS power receiver coil. The communication unit 58 can be configured to communicate control commands to the VAD 14 over the implantable controller-to-VAD connection cable 26. The communication unit 58 can also include a suitable wireless communication unit for receiving programming updates and/or for transmitting alarms, VAD operational data, and/or patient physiologic data to an external system monitor.

The controller 20 can be configured so that the haptic unit 60 is operated to generate a haptic alarm to alert the patient that power stored in the controller battery unit 52 and/or the TETS receiver battery unit 40 has dropped below a suitable minimum threshold so that the patient can take action to use the TETS power transmitter 24 to transmit power to the TETS power receiver 22 to recharge the controller battery unit 52 and/or the TETS power receiver battery unit. To guard against a prolonged latent failure of the haptic unit 60, the controller 20 can periodically command operation of the haptic unit 60 to determine whether the haptic unit 60 operated properly or is in a failed state. If the controller 20 determines that the haptic unit 60 is in a failed state, the controller 20 can communicate a suitable alarm indicating the failure of the haptic unit 60 via wireless communication by the communication unit 58.

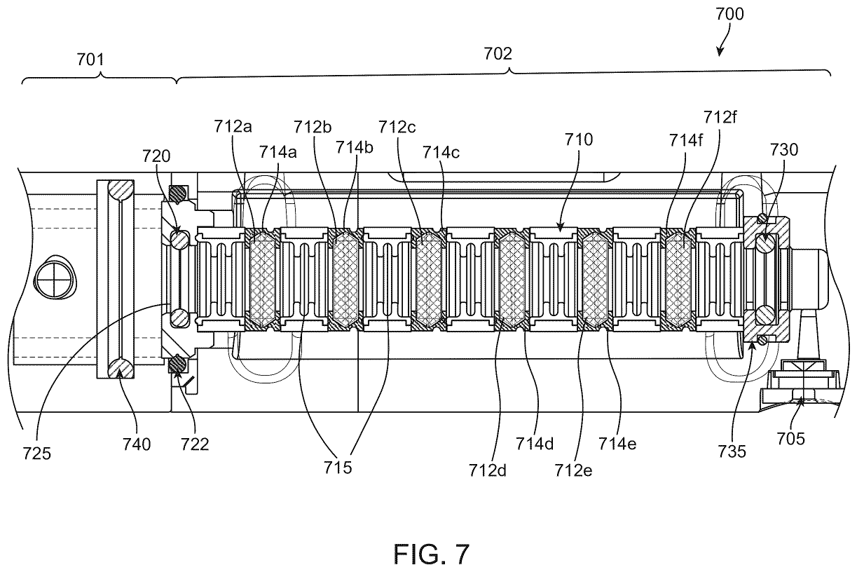

Referring to FIGS. 7 through 10, a configuration of the header 700 of the implantable controller 20 in FIG. 1 in combination with a lead receivable in the header 700 to electrically couple the implantable controller 20 with the VAD 14 and/or the TETS power receiver 22 for power supply and/or communication transmissions (e.g., data, control signals) is discussed in detail below. It can be appreciated by a person of the ordinary skill in the art that the header configuration and the implantable medical device herein (e.g., the controller 20) is presented by way of example to explain concepts of the present disclosure and does not limit the scope of the present disclosure. For example, the present disclosure may be equally applicable to other implantable medical devices having high power consumption demands (e.g., four or more conductors in the header) outside of blood circulation assist systems, such as implantable neurostimulators, pacemakers, and like devices. Further, the present disclosure may be applicable to other components of the blood circulation assist system, for example a bulkhead connector of the TETS power receiver or other implantable modules, for example the controller module discussed in U.S. Pat. No. 10,610,692, which is incorporated herein by reference in its entirety for all purposes.

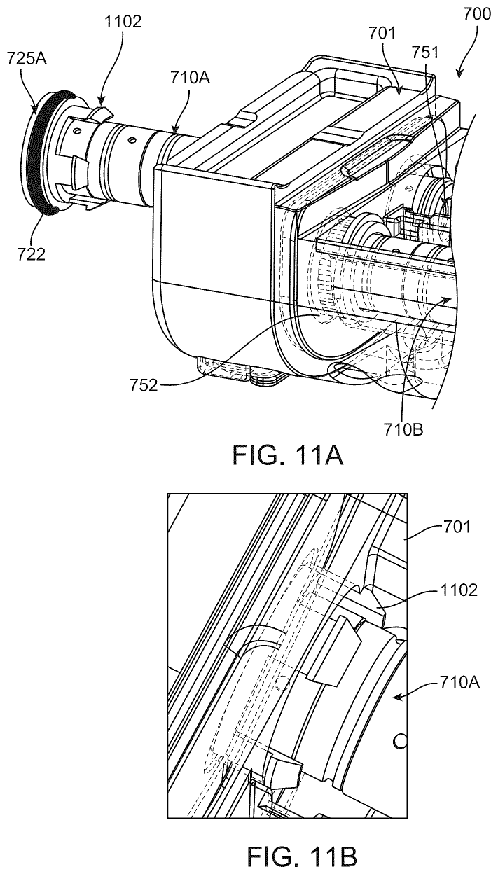

FIG. 7 illustrates a portion of the header 700 of the implantable medical device (e.g., the controller 20 in FIG. 1) coupled to the housing 70. The header 700 includes at least one receptacle connector stack 710 having a proximal end and a distal end. The receptacle connector stack 710 is disposed within the header 700 and configured to electrically couple with the VAD 14 and/or the TETS 22 as shown in FIG. 1. In many embodiments, the header 700 includes a cassette 701 and a header portion 702 distally extending from the cassette. The cassette 701 is configured to receive and lock the receptacle connector stack 710 in place within the header 700. The cassette 701 is a proximal portion of the header 700 and can be integrally formed with rest of the header 700. In some embodiments, the cassette 701 can be separately formed and attached to a proximal end of the header portion 702. Upon fully inserting the receptacle connector stack 710 through the cassette 701, the distally extending portion 702 receives and houses the receptacle connector stack 710. Various locking mechanisms for coupling the receptacle connector stack 710 at a proximal portion with the header 700 are discussed in detail below with respect to FIGS. 11-15.

In many embodiments, the header 700 includes two receptacle connector stacks (individually referred as 710A and 710B in FIGS. 11-15) spaced apart and electrically isolated from each other. For example, as shown in FIG. 1, a first receptacle connector stack (e.g., 710A) is configured to facilitate power supply and communication between the VAD 14 and the controller 20 via the implantable controller-to-VAD connection cable 26 and the second receptacle connector stack (e.g., 710B) is configured to facilitate power and communication between the TETS power receiver 22 and the controller 20 via the implantable TETS power receiver-to-controller connection cable 28.

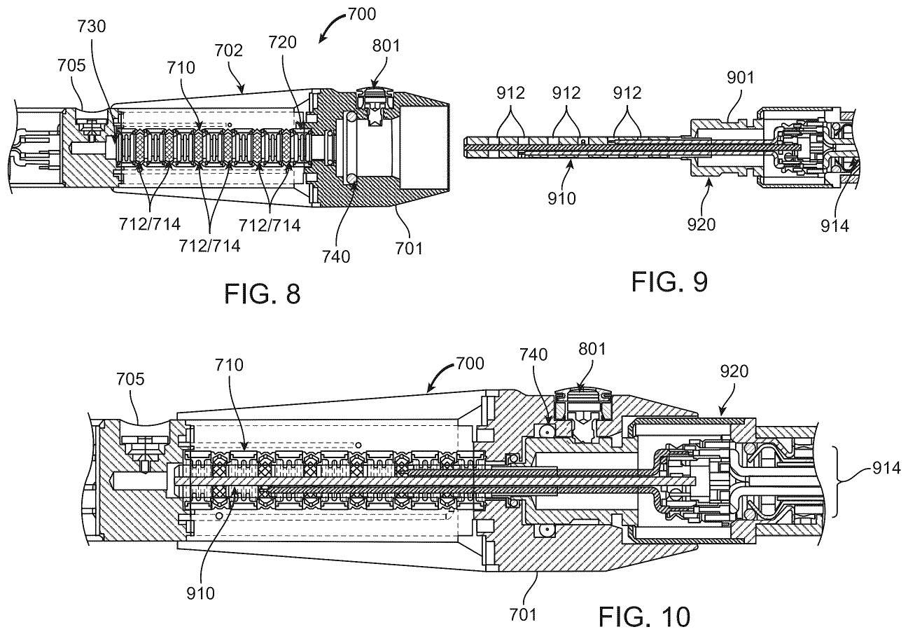

Referring to FIGS. 7 and 8, the receptacle connector stack 710 includes a plurality of electrical contacts 712a-712f (collectively referred to as 712) and a plurality of wiper seals 714a-714f (collectively referred to as 714). The plurality of contacts 712 are linearly spaced from each other by insulating members 715, where the insulating member 715 provides electrical isolation between the contacts 712 as well as supporting structure for additional components used for sealing against fluid ingress, electrical isolation, and/or assembling the receptacle connector stack 710 with the header 700. In the illustrated example, the receptacle connector stack 710 includes six electrical contacts, out of which two may be used for power transmission and four may be used for communication purposes (e.g., data, control signals, etc.). Each electrical contact (e.g., 712a) of the plurality of electrical contacts 712 is separated from adjacent electrical contacts by a corresponding wiper seal (e.g., 714a). The electrical contacts 712 may include, for example, ring shaped electrical contacts that are axially spaced from each other. The electrical contacts 712 may also include segmented electrical contacts disposed at an angle with respect to the longitudinal axis of the receptacle connector stack. Still further, the electrical contacts may comprise canted or cantilevered springs. The present disclosure is not limited by a type of electrical contact.

Each wiper seal (e.g., 714a) includes a tongue or a flap (e.g., wiper blade style) configured to isolate an electrical contact (e.g., 712a) from other electrical contacts. The wiper tongue (not illustrated) may be disposed on either sides of a corresponding electrical contact (e.g., 712a) to isolate from adjacent electrical contacts. The wiper tongue or flap is a compliant member that allows for adequate contact isolation while facilitating low lead insertion force. However, in many high powered medical implant applications, these wiper seals may not be adequate to isolate the electrical contacts, particularly the electrical contacts at the proximal end and the distal end of the receptacle connector stack 710. For example, the wiper tongue on a proximal end and/or a distal end of the receptacle connector stack 710 may be insufficient for inhibiting stray electrical currents from entering the electrical contacts and/or inhibiting fluid ingress from the outside environment.

To boost sealing performance and electrical isolation of the header, additional sealing elements are provided at the proximal end and the distal end, respectively, of the receptacle connector stack 710. A first sealing element 720 (also referred to as a proximal sealing element 720) is provided at a proximal end of the receptacle connector stack 710. Similarly, a second sealing element 730 (also referred to as a distal sealing element 730) is provided at a distal end of the receptacle connector stack 710. The first sealing element 720 and the second sealing element 730 can include O-rings. The sealing elements 720 and 730 are made of appropriate sealing material, such as implant grade silicone with durometers between 30-60 on the shore A scale, and sized (e.g., 0.115 to 0.145 inches for the inner diameters and 0.165 to 0.205 inches for the outside diameters) to allow easy assembly within the receptacle connector stack 710. If the sealing elements 720 and 730 are too small, fingers of the technician assembling the receptacle connector stack may become fatigued or additional assembly tools may be required. As such, the sealing elements 720 and 730 are sized such that an inner diameter and outer diameter of each O-rings is within a range of 0.115 to 0.145 inches for the inner diameters and 0.165 to 0.205 inches for the outside diameters to facilitate ease of assembly on the receptacle connector stack.

The first sealing element 720 is configured to inhibit fluid ingress and stray electrical currents at the proximal end of the receptacle connector stack 710 from an outside environment. The second sealing element 730 is configured to inhibit fluid ingress and stray electrical currents at the distal end of the at least one receptacle connector stack from the outside environment. The diameter size of the sealing elements 720 and 730 can also advantageously help in inhibiting fluid ingress. For example, a larger sealing element may provide better sealing performance compared to a smaller ring. The sealing elements 720 and 730 have diameters in the range of 0.115 to 0.375 inches for the inner diameters and 0.145 to 0.515 inches for the outside diameters that advantageously provide improved sealing performance and ease of assembly.

To facilitate improved seal handling and sizing capabilities, seal housings are provided. A proximal seal housing 725 is configured to receive the first sealing element 720, and a distal seal housing 735 is configured to receive the second sealing element 730. The proximal seal housing 725 and the distal seal housing 735 can be of similar size and construction for mass manufacturing and sized to easily assemble the respective sealing element. In some embodiments, the proximal seal housing 725 and the distal seal housing 735 are separately manufactured and assembled with the receptacle connector stack 710. In some embodiments, the proximal seal housing 725 and the distal seal housing 735 are integrally formed with the receptacle connector stack 710 to further minimize any gaps for fluid ingress and thus improve sealing performance of the header 700. The proximal seal housing 725 can further include a retention mechanism configured to engage with the receptacle connector stack 710 and the header 700. Example retention mechanisms are discussed with respect to FIGS. 11-15 below, in which the proximal seal housing 725 is individually referred to as 725A, 725B, 725C, 725D, and 725E, each having a particular type of retention mechanism.

Furthermore, an outer sealing element 722 is provided outside the receptacle connector stack 710 to seal any gaps between the cassette 701 and the proximal end housing 725. The outer sealing element 722 has a larger diameter than the proximal sealing element 720. The outer sealing element 722 is disposed within a groove formed in the cassette 701. The outer sealing element 722 can also be an O-ring that provides additional sealing to inhibit ingress of fluid from the proximal end. The outer sealing element 722 can be concentric with the first sealing element 720.

To evaluate improvements, experiments were performed with different seals. An example experimental setup included implantable devices with different seal configurations between leads and receptacle connector stacks (with and without proximal and distal booster sealing elements of the present invention) which were submerged in water for a period of time (e.g., 3 months or more). In the submerged state, each implantable device was monitored for leakage current from the electrical contacts (e.g., at the distal end and the proximal end) of the receptacle connector stack and the lead. Particularly, in each of these experiments, contact-to-contact leakage current was measured from readings taken from within the header between all contacts and internal contact-to-environment leakage current was measured from readings taken from all contacts within the header to outside environment. Devices without booster sealing elements exhibited contact-to-contact and contact-to-environment leakage current of greater than 1 μA, which led to eventual failure of these devices due to high leakage current. In contrast, experimental devices configured with the proximal seal 720 and the distal seal 730 according to the present disclosure exhibited negligible leakage current. Notably, no contact-to-contact or contact-to-environment leakage current greater than luA was detected, which clearly showed improved sealing performance of the booster sealing components.

In many embodiments, the wiper seal (e.g., 714a) includes wiper tongues extending from an inner surface toward an electrical contact (e.g., 712a). For example, the wiper seals 714 have a hollow cylindrical shape or a ring shape with a tongue extending from an inner diameter of the ring or the cylinder. When the receptacle connector stack 710 receives a lead (e.g., a lead 910 shown in FIG. 9), the wiper tongue moves backward (e.g., like wiper blades) relative to an axially inserting direction. Upon completing the insertion of the lead, the wiper tongue tries to retain its original position. When returning to the original position, the wiper tongue can push on the lead (e.g., the lead 910) causing the lead to axially displace or misalign with the electrical contacts 712 of the receptacle connector stack 710. In other words, the interaction of the receptacle connector stack 710 and the lead 910 can often create a hydrostatic effect or a piston-cylinder effect where the lead 910 may move relative to desired axial insertion location within the receptacle connector stack 710. This phenomenon can be described as the “bounce back” effect. For example, the lead 910 can experience approximately 0.010 inches to 0.15 inches of axial shift due to this bounce back effect.

In many embodiments, a bounce back reducing feature is provided to reduce or prevent axial displacement from a desired position or misalignment between the lead 910 and the connector stack 710. As shown in FIG. 7, a bounce back reducer 740 is disposed at the proximal end of the receptacle connector stack 710 to reduce bounce back effects experienced by lead insertion. In many embodiments, the bounce back reducer 740 is an O-ring. The bounce back reducer 740 has a larger diameter than a diameter of the proximal sealing element 720. The bounce back reducer 740 can be installed in the cassette 701 of the header 700. The inner diameter of the bounce back reducer 740 is in a range from about 0.275 to about 0.375 inches and the outer diameter of the bounce back reducer 740 is in a range from about 0.335 to about 0.515 inches.

In many embodiments, the header 700 may further includes a septum or vent 705 located distally from the distal sealing element 730 of the receptacle connector stack 710 to prevent hydrostatic locking between the implantable lead 910 and the receptacle connector stack 710. The septum 705 may provide another opening for ingress of fluid and stray current. In this regard, the distal sealing element 730 can advantageously inhibit ingress of fluid and any stray current from the septum.

FIG. 8 illustrates the header 700 with the receptacle connector stack 710 disposed therein and configured to receive a lead 910 shown in FIG. 9. The cassette 701 receives and retains the receptacle connector stack 710 to the header 700. The receptacle connector stack 710 extends axially in the header 700 such that the electrical contacts 712 and the wiper seals 714 are completely within the distally extending portion 702 of the header 700. The cassette 701 includes a locking means 801 (e.g., set screw) configured to securely engage the lead 910 within the receptacle connector stack 710. For example, the locking means 801 is a screw that can be adjusted (e.g., tightened or loosened) to engage or disengage the lead 910. The lead 910 is attached to a proximal lead housing 920. The lead housing 920 includes a slot or a groove 901 configured to receive the locking means 801, as shown in FIG. 10. The locking means 801 such as the screw can be tightened to securely engage and prevent axial movement of the lead 910 relative to the receptacle connector stack 710 once the lead 910 is inserted therein.

The lead 910 is an elongated shaft including a plurality of electrical contacts 912 axially spaced from each other. The relative location of the electrical contacts 912a-912f correspond to locations of the plurality of electrical contacts 712a-712f of the receptacle connector stack 710. Upon assembly, the electrical contacts 912 are electrically coupled to corresponding electrical contacts 712. In the example shown, each of electrical contacts 912 is a ring contact. An electrical contact of 912 can be connected to an electrical wire inside the lead 910 and extending from within the lead 910 to an electrical connector or interface 914 in the proximal lead housing 920. The electrical connector 914 include wires from the lead 910 coupled to or grouped into or integrated within the implantable controller-to-VAD connection cable 26 or the TETS power receiver-to-controller connection cable 28 to electrically couple the lead 910 to VAD 14 or TETS power receiver 22.

Upon inserting the lead 910 into the receptacle connector stack 710, as shown in FIG. 10, the electrical connectors 912 are electrically coupled to corresponding electrical connectors 712 of the receptacle connector stack 710 thereby further coupling with the electrical connector 914. The electrical connection between connectors 712, 912, and 914 facilitates power and communication transmission between the implantable controller 20 via the header 700 to the VAD 14, and/or TETS power receiver 22 (shown in FIG. 1). The connector assembly including the receptacle connector stack 710 coupled to the lead 910 in the header 700 is electrically coupled with other electronic components of the implantable controller 20 hermetically sealed in the housing 70 of the controller 20. In some embodiments, feedthrough assemblies between the header 700 and housing 70 of the controller 20 facilitate electrical coupling of the connector assembly to the other electronic components of the controller 20 (e.g., the processor 56 configured to control VAD speed, the memory 54 to store data, the rechargeable battery 52 to store power received from the TETS power receiver 22, etc., as shown in FIG. 6 above).

As mentioned earlier, inserting the lead 910 into the receptacle connector stack 710 causes a bounce back effect. As shown in FIG. 10, the bounce back reducer 740 engages with the circumference of the proximal lead housing 920 and secures the lead 910 in place with respect to the cassette 701 thereby reducing any bounce back effect that the lead 910 experiences from the wiper seals 714 of the receptacle connector stack 710. The bounce back reducer 740 can be an O-ring which can inhibit ingress of fluid from an outside environment through the lead 910 into the receptacle connector stack 710 at the proximal end. Additionally, the distal sealing element 730 provides sealing of the electrical contacts at the distal end and the proximal sealing element 720 provides another layer of sealing of the electrical contacts at the proximal end from the outside environment. Thus, the distal and proximal sealing element 730, 720 together with the bounce back reducer 740 can advantageously enhance the sealing performance of the implantable medical device.

Referring now to FIG. 11A, a snapping means for attaching the receptacle connector stack 710A and/or 710B to the header 700 is illustrated. The header 700 includes internal channels to receive a first receptacle connector stack 710A and a second receptacle connector stack 710B, respectively. The internal channels are spaced from each other and electrically isolated.

11B shows an enlarged portion of a snapping element of the snapping means. Specifically, the snapping element 1102 is provided at the proximal end of the first receptacle connector stack 710A (and similarly on the second receptacle connector stack 710B). In many embodiments, the snapping means is provided on a proximal seal housing 725A. As shown, the proximal seal housing 725A is configured to include the snapping elements 1102. The proximal housing 725A is coupled to the receptacle connector stack 710A at the proximal end. The receptacle connector stack 710A can be inserted through a channel 751 in the cassette 701 into a distally extending portion 702 within the header 700.

The snapping elements 1102 are cantilevered elements with deflectable flanges at a distal end. Upon inserting the receptacle connector stack 710A through the channel 751, the flanges of the snapping elements 1102 engage with an inner surface of the header 700 thereby securing the receptacle connector stack 710A to the header 700. The proximal housing 725A also mounts the proximal sealing element 720 (as shown in FIG. 7) and the outer sealing element 722 disposed proximal to the snapping element 1102 and co-axial with the proximal sealing element 720. Further, a groove may be provided to receive the outer sealing element 722. As such, upon assembly, the proximal sealing element 720 (seen in FIG. 7) and the outer sealing element 722 inhibit ingress of fluid through the proximal end of the receptacle connector stack 710A into the header 700.

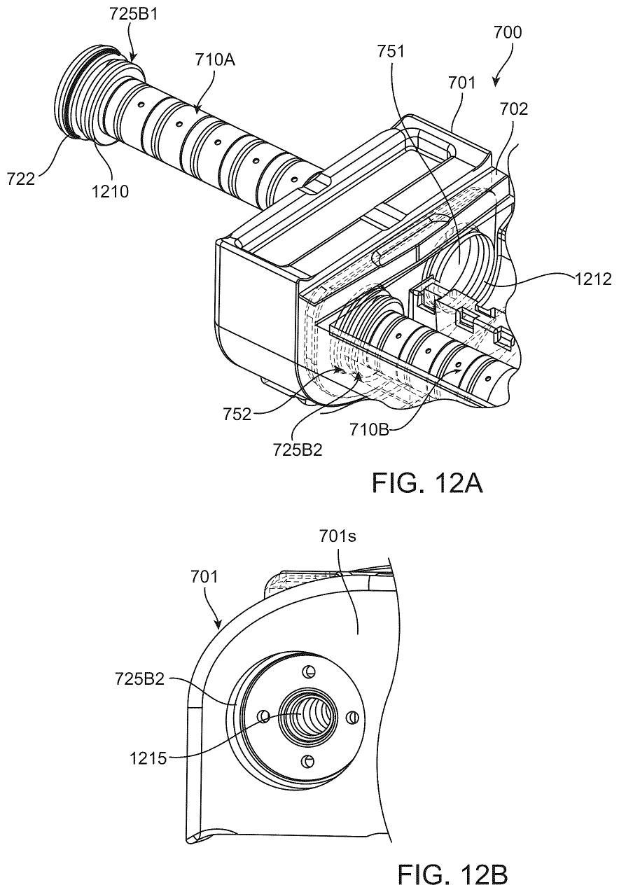

FIG. 12A and FIG. 12B illustrate a retention mechanism comprising a thread fit used to retain the receptacle connector housing 710A with the header 700. Threads 1210 are formed at a proximal end portion of the receptacle connector stack 710A and configured to engage with corresponding threads 1212 formed in the channel 751 of the cassette 701 to secure the receptacle connector stack 710A to the header 700. In many embodiments, the threads 1210 are formed on an outer periphery of the proximal seal housing 725B1 which is fixedly attached or integrally formed at the proximal end portion of the receptacle connector stack 710A. The proximal seal housing 725B1 is also configured to receive the proximal sealing element 720 (seen in FIG. 7) and the outer sealing element 722. For example, the proximal seal housing 725B1 includes grooves for mounting the sealing elements 720 and 722 (e.g., O-rings). The sealing elements 720 and 722 are disposed proximal to the threads 1210 to provide sealing between the header 700 and the receptacle connector stack 710A and inhibit ingress of fluid from an outside environment. As such, when the proximal seal housing 725B1 is threadingly coupled to the channel 751 of the cassette 701, the sealing elements 720 and 722 inhibit ingress of fluid into the header 700 or the receptacle connector stack 710A.

FIG. 12A also illustrates a second receptacle connector stack 710B having similar construction as the first receptacle connector stack 710A. The first and the second receptacle connectors 710A and 710B are spaced apart from each other and disposed in hollow channels in the header 700. The second receptacle connector stack 710B is threaded to a second channel 752 formed in the cassette 701. For example, a second proximal seal housing 725B2 includes external threads, similar to the threads 1210 of the first proximal seal housing 725B1, that engage with threads of the second channel 752. As the hollow channels of the header 700 are electrically isolated, electrical coupling between the receptacle stacks 710A and 710B is achieved via the electrical contacts and electronic components disposed in the controller 20.

FIG. 12B further illustrates a front view showing the second proximal seal housing 725B2 (and similarly applicable for the proximal seal housing 725B1) assembled in the cassette 701. As shown, upon assembly, an end surface of the second seal housing 725B2 is flush with outer surface 701s of the cassette 701 or the end surface of the second seal housing 725B2 is inside the channel 752 of the cassette 701 to provide a seamless outer surface of the header. Also as shown, an inner portion 1215 of the second proximal seal housing 725B2 (and similarly the proximal seal housing 725B1) may include an attachment means configured to couple the second proximal seal housing 725B2 to the receptacle connector stack 710B. In many embodiments, the inner portion 1215 may include threads (in FIG. 12B), slot and pin, adhesive, or other removable or fixed attachment means. In some embodiments, the end surface of the second seal housing 725B2 may also include assembling feature (e.g., holes, or other means) for inserting and removing the receptacle connector stack 710B from the header 700.

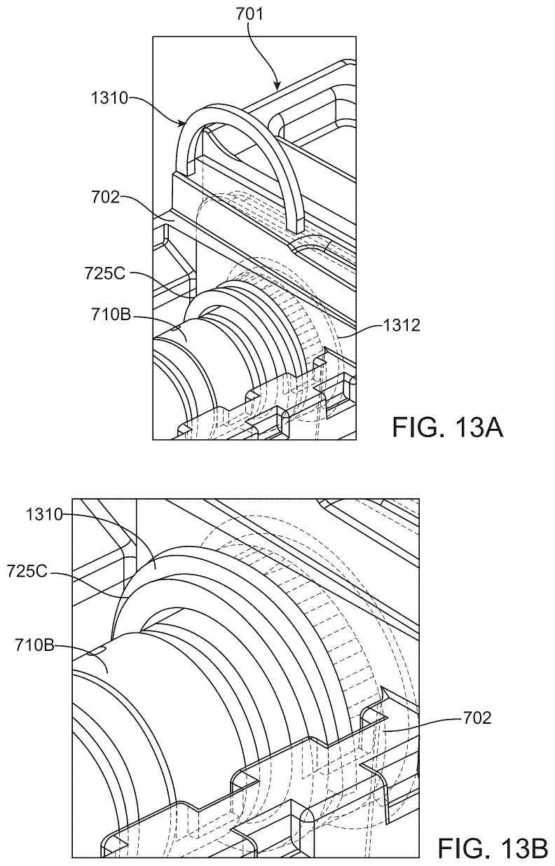

FIG. 13A illustrates a retaining ring 1310 used for coupling the receptacle connector stack 710B to the header 700. FIG. 13B illustrates an enlarged portion of the proximal end of receptacle connector stack 710B coupled with the retaining ring 1310. The retaining ring 1310 is a semicircular ring shaped to fit a proximal portion of the receptacle connector stack 710B and abut an inner surface of the header 700 so that the receptacle connector stack 710B is securely engaged in the channel of the header 700. In many embodiments, the proximal seal housing 725C is configured to receive the retaining ring 1310 while being coupled to the receptacle connector stack 710B. The proximal seal housing 725C includes a retainer groove 1312 corresponding to the shape and size of the retaining ring 1310. For example, the retainer groove 1312 may be semicircular in shape and of same thickness as the retaining ring 1310. The retainer groove 1312 is formed on a portion of the proximal seal housing 725C that is inside the header 700 to facilitate coupling with an inner surface of the header 700.

To assemble the retaining ring 1310, the receptacle connector stack 710B is inserted in the cassette 701 of the header 700. The distally extending portion 702 of the header 700 is partially open (e.g., top portion) to enable placing the retaining ring 1310 within the retainer groove 1312. Once the retaining ring 1310 is assembled, as shown in FIG. 13B, the open portion of the distally extending portion 702 of the header 700 can be closed, for example by overmolding header material (e.g., epoxy) to enclose the header 700 with the receptacle connector stack 710B disposed therein.

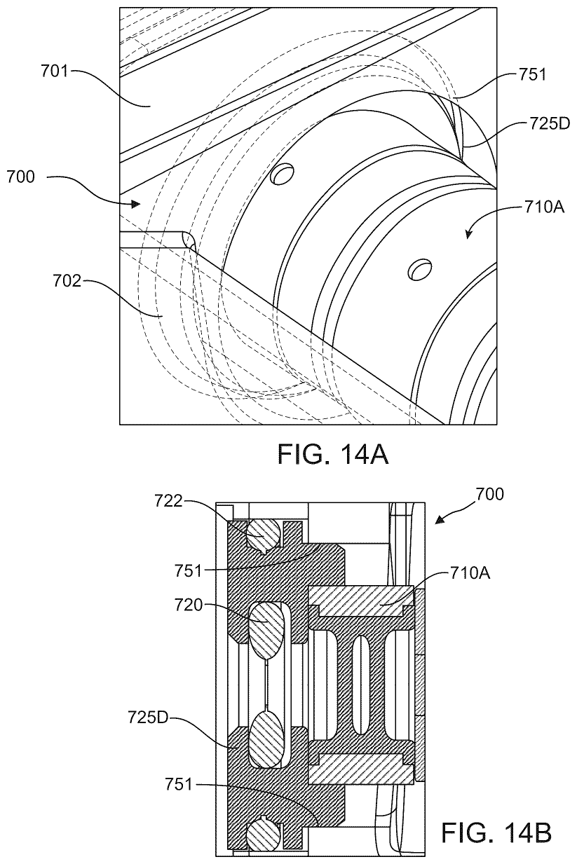

FIG. 14A illustrates a press fit or tight fit used for coupling the receptacle connector stack 710A to the header 700 and FIG. 14B illustrates a cross-section of a press fitted receptacle connector stack 710A. The press fit is provided by dimensioning an outer periphery of the receptacle connector stack 710A to tightly fit in the channel 751 of the cassette 701 of the header 700. In many embodiments, to facilitate the press fit, the proximal seal housing 725D is attached at the proximal end of the receptacle connector stack 710A and an outer periphery of the proximal seal housing 725D is sized to tightly engage with the inner periphery of the channel 751. In the example shown, a distal portion of the proximal sealing element 725D has an outer diameter slightly (e.g., 0.1%-5%) larger than an inner diameter of the channel 751 to facilitate the press fit.

The proximal seal housing 725D also mounts the outer sealing element 722 and the proximal sealing element 720, as shown in FIG. 14B. The sealing elements 722 and 720 are located proximal to the header 700. As such, when the distal portion of the proximal seal housing 725D tightly engages with the channel 751 to secure the receptacle connector stack 710A to the header 700, the proximal sealing elements 720 and 722 inhibit ingress of fluid from the proximal end providing improved sealing performance in addition to some sealing achieved by the press fit.

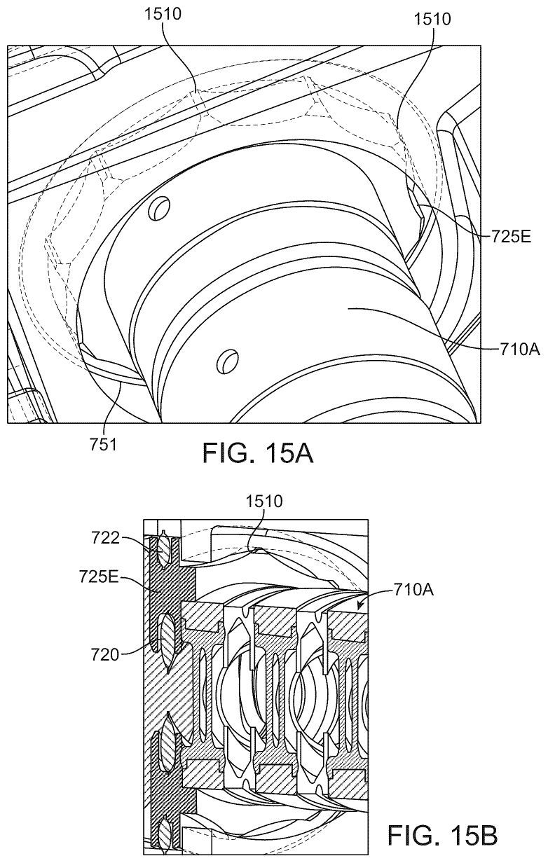

FIG. 15A illustrates a crush rib fit used for coupling the receptacle connector stack 710A to the header 700 and FIG. 15B illustrates a cross-section of a crush rib fitted receptacle connector stack. The crush rib fit is provided by ribs 1510 formed on an outer periphery of the receptacle connector stack 710A. The ribs 1510 are configured to crush or dig into an inner periphery of the channel 751 upon engaging with the channel 751 of the cassette 701. In many embodiments, to facilitate the crush rib fit, the proximal seal housing 725E is attached at the proximal end of the receptacle connector stack 710A and an outer periphery of the proximal seal housing 725E includes the ribs 1510. In the example shown, a distal portion of the proximal sealing element 725E has an outer diameter defined by the ribs 1510. The outer diameter of the ribs 1510 is slightly larger (e.g., 0.1%-5%) than an inner diameter of the channel 751 to facilitate tight engagement of the ribs into the inner periphery of the channel 751.

Furthermore, the proximal seal housing 725E also mounts the outer sealing element 722 and the proximal sealing element 720, similar to that shown in FIG. 14B. The sealing elements 722 and 720 are located proximal to the header 700. As such, when the ribs 1510 at the distal portion of the proximal seal housing 725E are engaged with the channel 751 to secure the receptacle connector stack 710A to the header 700, the proximal sealing elements 720, 722 inhibits ingress of fluid from the proximal end thereby improving sealing performance in addition to some sealing achieved by the crush rib fit.

Conventional implantable leads are typically short and rigid and may use up to three electrical lines of conduction within a constrained diameter of approximately 3.2 mm. However, as discussed above, several implantable medical devices that are high powered, like the VAD controller of the present invention, can require six electrical lines of conductions including a power, a ground, and a communication line, each line having a redundancy for improved operational safety of the implantable device and patient. Adding more lines of conduction within a small and constrained diameter (e.g., less than 3.2 mm) is not a trivial task and presents several challenges related to manufacturing and maintaining structural integrity of the lead. For example, stacking conduction lines on top of each other within the approximately 3.2 mm diameter results in a long but skinny in girth male lead plug that may have reliability issues.

According to the present disclosure, an implantable lead is configured to accommodate six full electrical lines of conduction while achieving improved strength within a desired lead diameter. In particular, the robust male lead plugs of the present invention coaxially stack multiple conductors that can be linearly spaced from each other and include an elongate rigid strength member for added rigidity and stiffness. The conductors can be ring electrical contacts. The implantable lead can be axially modular to accommodate one or more conduction lines. For example, the conduction lines can be flexible electrical wires of different lengths, each of which can transmit signals received by the electrical contacts. The multiple electrical contacts and the conduction lines are configured such that the implantable lead plug does not require tightening of a set screw or other external activation means to electrically activate one or more conduction lines. The conduction lines can be flexible electrical wires, rigid and flat electrical metal lines, or other electrical conduction lines. The conduction lines are referred as electrical wires as an example to explain the concepts of the present disclosure without limiting the scope of the present disclosure. The implantable lead can accommodate flexible electrical wires of specified diameter to satisfy conduction and heating specifications related to an implantable medical device. In many embodiments, the implantable lead can include a stem (e.g., made of titanium or other biocompatible material) on one end for handling the lead and a shoulder on the stem can be provided as an additional sealing interface.

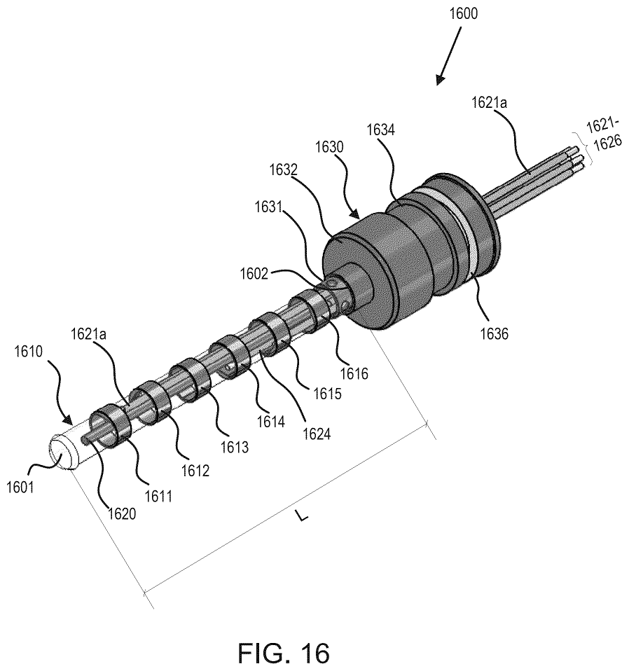

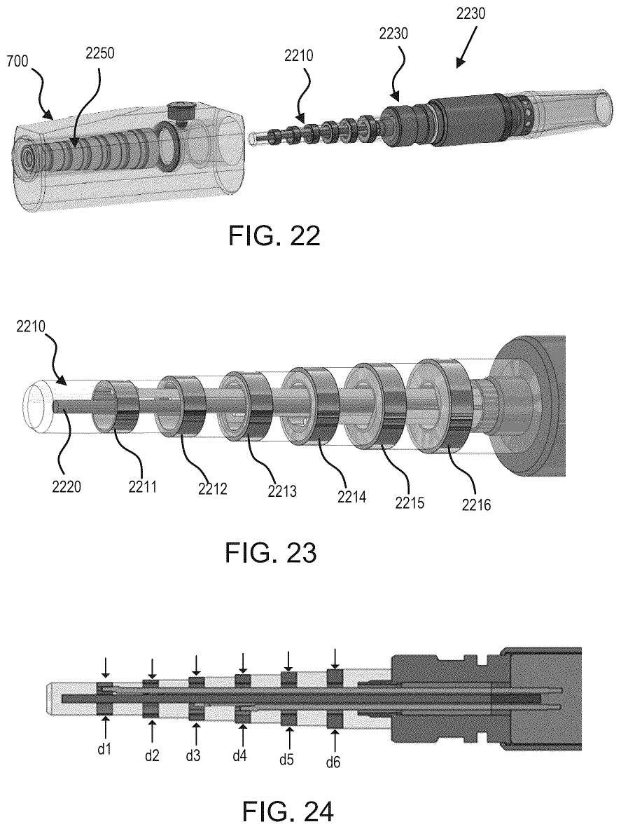

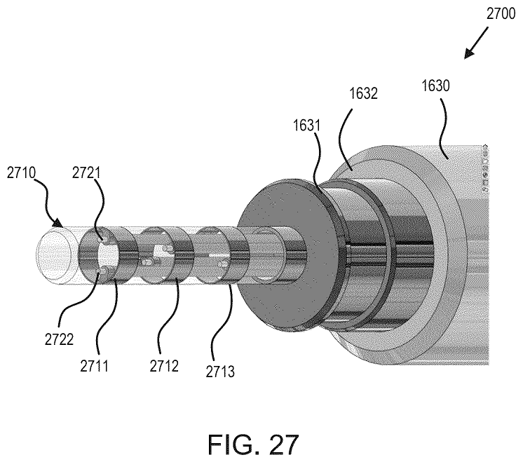

FIGS. 16-27 illustrate different configurations of the robust implantable lead of the present invention. For example, FIGS. 16-18 illustrate the implantable lead with six electrical contacts with six electrical wires. FIG. 22-26 illustrate an implantable lead with four electrical contacts with six electrical wires. FIG. 27 illustrates an implantable lead with three electrical contacts with six electrical wires.

Referring now to FIG. 16 illustrates a perspective view of an implantable lead 1600, in accordance with many embodiments. The implantable lead 1600 includes an elongate shaft 1610, a plurality of electrical contacts 1611-1616, and a plurality of electrical wires 1621-1626. The implantable lead 1600 can further include a strength member 1620 to improve strength and rigidity of the lead, and a stem 1630 to improve handling and coupling of the lead. For example, a proximal portion of the elongate shaft 1610 can be coupled to the stem 1620. To manufacture the lead, several components including the elongate shaft 1610, the electrical wires 1621-1626, and/or the strength member 1620 can be overmolded. Overmolding over long wires and/or the strength members can present manufacturing challenges due to flowability of a molding material and tendency of the wires and the strength member to move while the molding material is being cured or hardened. As such, lengths and sizes of the wires and the strength members discussed herein can be advantageous from a manufacturing perspective in addition to meeting electrical conduction or signal transmission specifications related to implantable devices.

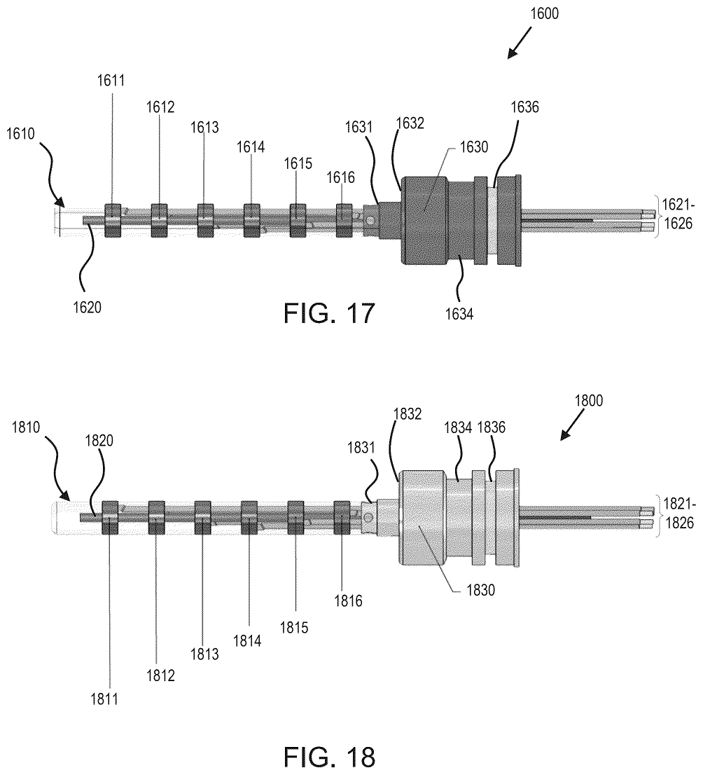

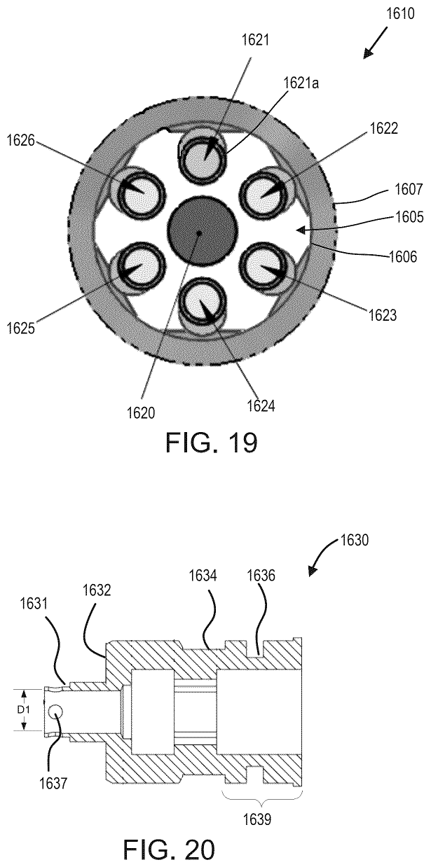

The elongate shaft 1610 has a distal end 1601, a proximal end 1602, and a uniform diameter between the distal end 1601 and the proximal end 1602. The distal end 1601 may be chamfered so that the elongate shaft 1610 properly aligns and rests in a receiving member such as a receptacle connector stack. The elongate shaft 1610 can have a diameter of approximately 3.2 mm. For example, as shown in FIG. 19, the elongate shaft 1610 has an inner diameter 1606 and an outer diameter 1607. In an example, the outer diameter 1607 can be 3.2 mm and the inner diameter 1606 can be less than 3.2 mm (e.g., 3 mm, 2.9 mm or less). The electrical wires 1621-1622 are coupled (e.g., soldered) at the inner diameter 1606 of the elongate shaft 1610. An overmold thickness over the outer wire diameters can be approximately 0.5 mm or less. Referring to FIG. 16, a length L of the elongate shaft 1610 can be in a range from 31 mm to 33 mm. It can be understood that the present disclosure is not limited to a particular length and other lengths of elongate shaft 1610 are possible. For example, the length L can be in a range from 6 mm to 50 mm. The elongate shaft 1610 can be made of insulating materials 1605 (see FIG. 17) that are moldable. The insulating materials can include, but not limited to, polyetheretherketone (PEEK), thermoplastic polyurethanes (TPU) like tecothane, estane, or polysulfones. For example, the elongate shaft 1610 can be made of resins (e.g., polyether-based thermoplastic polyurethanes) that are corrosion resistance and exhibit biostability over a wide range of hardness.

The plurality of electrical contacts 1611-1616 are disposed on an outer surface of the elongate shaft 1610. The plurality of electrical contacts 1611-1616 are separated by a molded insulative material for electrical isolation. For example, the electrical contacts 1611-1616 are disposed on an outer surface of the elongate shaft 1610 and axially spaced from each other. The spacing and insulative material therebetween provides electrical isolation between each of the contacts. In one embodiment, the plurality of electrical contacts includes a first electrical contact 1611 and a second electrical contact 1612 for power transmission, a third electrical contact 1613 and a fourth electrical contact 1614 for communication, and a fifth electrical contact 1615 and a sixth electrical contact 1616 serving as ground. In one embodiment, each of the electrical contacts 1611-1616 can be a ring contact. The ring contacts are coaxial and linearly spaced from each other along the elongate shaft 1610. Each of the ring contact can be of approximately same diameter and axial length. However, the present disclosure is not limited to a particular type of contact or a particular axial length of the contact. Each of electrical contacts 1611-1616 can be electrically coupled to a respective wire of the wires 1621-1626 within the elongate shaft 1610. For example, the wires can soldered, glued, screwed, or directly attached to the respective contacts The electrical contacts 1611-1616 and the electrical wires 1621-1626 are made of corrosion resistant and electrically conductive material that can be safely implanted in a patient. For example, the electrical contacts and the wires are made of platinum iridium or a nickel-cobalt base alloy of a multiphase alloy.

In one embodiment, as shown in FIGS. 16 and 17, the first and the second electrical contacts 1611 and 1612 for power transmission are disposed at a distal portion of the elongate shaft. The fifth and the sixth electrical contacts 1615 and 1616 serving as ground are disposed at a proximal portion of the elongate shaft. The third and the fourth electrical contacts 1613 and 1614 for communication are disposed between the proximal portion and the distal portion of the elongate shaft. However, other variations or combinations of electrical contacts and its corresponding functions are possible without limiting the scope of the present disclosure.

The plurality of wires 1621-1626 are electrically coupled to the plurality of electrical contacts and extending axially within the elongate shaft 1610. In many embodiments, the plurality of wires 1621-1626 may be radially disposed approximately 60 degree from each other (see FIG. 19). In one embodiment, the plurality of wires includes a first wire 1621 and a second wire 1622 electrically coupled to the first electrical contact 1611 and the second electrical contact 1612, respectively; a third wire 1623 and a fourth wire 1624 electrically coupled to the third electrical contact 1613 and the fourth electrical contact 1614, respectively; and a fifth wire 1625 and a sixth wire 1626 electrically coupled to the fifth electrical contact 1615 and the sixth electrical contact 1616, respectively. Accordingly, a length of the sixth wire 1626 is shortest, while a length of the first wire 1621 is longest. Each of the plurality of wires 1621-1626 have a diameter of approximately 0.3 mm to 1 mm and a varying length within the elongate shaft 1610. The electrical wires can have an exposed length from the stem in a range from 1 mm to 50 mm to establish electrical connection with connectors or medical devices. In many embodiments, each of the wires 1621-1626 further includes an insulation cover (e.g., 1621a) disposed over each wire except at electrical connection with respective electrical contacts 1611-1616. The insulation cover provided additional electrical insulation between wires and the strength member 1620 within the elongate shaft 1610 and insulation when the wires 1621-1626 are exposed from the stem 1630.

The strength member 1620 is disposed axially within the elongate shaft 1610. The strength member 1620 and the plurality of wires 1621-1626 are electrically isolated from each other and are constrained within a diameter of the elongate shaft. In many embodiments, the plurality of wires are radially disposed approximately 60 degree from each other and circumferentially around the strength member 1620 (see FIG. 19). The strength member 1620 has a diameter in a range from 0.5 to 1.5 mm and a length less than a length L of the elongate shaft 1610. For example, a length of the strength member 1620 may be approximately 2.5 mm to 3 mm shorter than the length L of the elongate shaft 1610. In some embodiments, the strength member 1620 can extend partially along a length of the elongate shaft 1610. For example, the strength member 1620 can extend from the first electrical contact 1611 at the distal portion to the sixth electrical contact at the proximal portion of the elongate shaft 1610. In another example, the strength member 1620 can further extend into and project from proximal end of the stem 1630. In many embodiments, the strength member 1620 can be made of biocompatible material such as titanium or other rigid materials that are corrosion resistant. A titanium strength member does not interfere with electrical communication or signals carried by the wires 1621-1626 as these are isolated from the strength member 1620 so it is not electrically conductive.

FIG. 20 illustrates an example cross-section of the stem 1630, according to many embodiments. The stem 1630 can be a hollow cylinder with a distal portion 1631 configured to receive the proximal portion of the elongate shaft 1610. Accordingly, the stem 1630 can also be referred as a proximal lead housing (e.g., the lead housing 920 in FIG. 9). The distal portion 1631 of the stem 1630 has an inner diameter D1 approximately equal to an outer diameter of the proximal portion of the elongate shaft 1610 to fittingly receive the proximal portion of the elongate shaft 1610. The distal portion 1631 may further include a hole 1637 for securely coupling the elongate shaft 1610 to the stem 1630. For example, the hole 1637 can be used to receive adhesive, a fastener, or other fastening means to couple the elongate shaft 1610. In many embodiments, the hollow cylinder may include stepped inner diameters with diameters greater than the diameter D1 for accommodating additional elements such as an electrical connector at the proximal portion 1639 of the stem 1630.

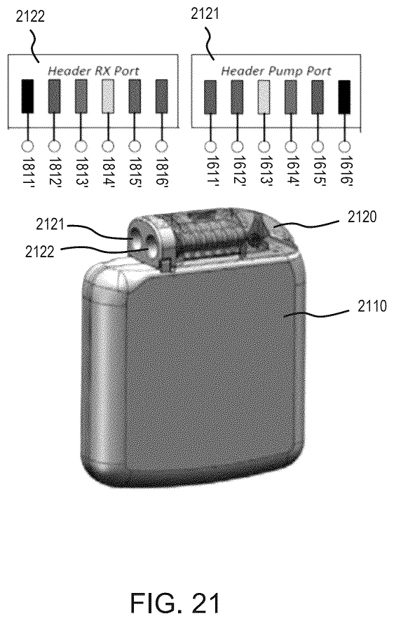

The stem 1630 includes a shoulder 1632 to receive a sealing element at the distal portion 1631, and a groove 1634 configured to receive a locking assembly (e.g., a set screw) for securely coupling the implantable lead 1600 to a header assembly of a second implantable medical device (e.g., see the header assembly 2120 of the implantable controller 2110 in FIG. 21 or a similar implantable controller 20 with the header 700 in FIG. 1). In many embodiments, the stem 1630 may further include another a groove 1636 configured to receive another sealing element (e.g., an O-ring) to prevent ingress of fluid and/or improve handling of the lead from the proximal portion of the stem 1630.

In many embodiments, the stem 1630 can be dimensioned to fit different types of implantable leads discussed herein. For example, a stem 1830 (in FIG. 18) has substantially similar construction as 1630 with inner diameter of the stem 1830 being configured to receive the implantable lead 1800. For example, the stem 1830 also includes a distal portion 1831 to receive the distal portion of the elongate shaft 1810, a shoulder 1832 and grooves 1834 and 1836 (similar to elements 1632, 1634, and 1636). Similarly, a stem 2230 (in FIG. 22) can be configured to receive a proximal portion of a stepped diameter elongate shaft.

In one embodiment, as shown in FIGS. 16 and 17, the implantable lead 1600 can be used as a ventricular assist device (VAD) lead (e.g., a lead coupled to the VAD 14 in FIG. 1). In many embodiments, another implantable lead (having similar construction as the lead 1600) can be a transcutaneous energy transmission system (TETS) receiver lead (e.g., a lead coupled to the TETS receiver 22 in FIG. 1). The transcutaneous energy transmission system (TETS) receiver lead is also referred as a second implantable lead 1800 (e.g., see FIG. 18) and further discussed in detail below.

The second implantable lead 1800 can have a similar construction as the implantable lead 1600. In one embodiment, as shown in FIG. 18, the elongate shaft 1810 of second implantable lead 1800 has a larger diameter than a diameter of the elongate shaft 1610 of the implantable lead 1600 (also referred as the first implantable lead 1600. The larger diameter ensures that the implantable lead 1600 and the second implantable lead 1800 are correctly assembled within respective receptacle connector stacks of a header assembly of an implantable controller. An example implantable controller 2110 is shown in FIG. 21.

The implantable controller 2110 can include a first receptacle connector stack 2121 configured to receive the first implantable lead 1600 to electrically couple the implantable heart pump (e.g., the VAD 14 in FIGS. 1 and 2) to the implantable controller 2110, and a second receptacle connector stack 2122 configured to receive the second implantable lead 1800 to electrically couple the TETS receiver (e.g., the TETS receiver 22) to the implantable controller 2110. The first receptacle connector stack 2121 is spaced from the second receptacle connector stack 2122 within the header assembly 2120 and electrically isolated from each other. Each receptacle connector stack has specific electrical connections to receive a particular lead. For example, the first receptacle connector stack 2121 includes electrical contacts 1611′-1616′ corresponding to respective contacts 1611-1616 to receive the first implantable lead 1600, and the second receptacle connector stack 2122 includes electrical contacts 1811′-1816′ corresponding to respective contacts 1811-1816 to receive the second implantable lead 1800. The contacts 1611′-1616′ and 1811′-1816′ are further connected to circuitry in the implantable controller 2110 to control the implantable devices. The leads 1600 and 1800 cannot be interchangeably assembled within the header assembly 2120. Hence, the implantable leads 1600 and 1800 with different diameters sizes can be provided to ensure that the leads are not mistakenly assembled in an incorrect receptacle connector stack and to prevent any electrical damage to the implanted devices (e.g., TETS receiver).

Referring back to FIG. 18, the second implantable lead 1800 (similar to the implantable lead 1600) includes a second elongate shaft 1810, a second set of six electrical contacts 1811-1816 coaxially mounted on the outer surface of the second elongate shaft 1810, a second set of six wires 1821-1826 disposed within the second elongate shaft 1810. Each of the wires 1821-1826 can be electrically coupled to a respective electrical contact of the second set of six electrical contacts 1811-1816 at one end and the TETS receiver at an opposite other end. A second strength member 1820 can be disposed within the second elongate shaft 1810 and electrically isolated from the second set of six wires 1821-1826.

In one embodiment, arrangement of the electrical contacts of the implantable lead 1600 can be different from that of the second implantable lead 1800. For example, the second set of six electrical contacts 1811-1816 of the second implantable lead 1800 includes a first electrical contact 1811 can serve as a first volt (e.g., 5V) supply to power the controller 2110 and can be disposed at a distal portion of the second implantable lead 1800. A second electrical contact 1812 can serve as a ground and disposed adjacent and proximal the first electrical contact 1811. A third electrical contact 1813 can serve as a power terminal and disposed adjacent and proximal the second electrical contact 1812. A fourth electrical contact 1814 can serve as a communication line to communicate with TETS receiver and disposed adjacent to the third electrical contact 1813. A fifth electrical contact 1815 can serve as another ground and disposed adjacent to and proximal the fourth electrical contact 1814. A sixth electrical contact 1816 can server as another power terminal and disposed at a proximal portion of the second implantable lead 1800.

FIGS. 22-24 illustrate a stepped implantable lead 2200, according to another embodiment. The stepped implantable lead 2200 can operate in a similar manner as the implantable lead 1600 or 1800. In particular, the stepped or cone style construction of the elongate shaft can provide improved rigidity, strength and improved packing of electrical wires of different sizes. For example, an electrical wire for conducting power can be larger and disposed at a proximal portion of the elongate shaft. The stepped implantable lead 2200 can be coupleable to a corresponding stepped receptacle connector stack 2250, which can be disposed within a header assembly (e.g., the header 700) of an implantable device. The stepped diameter lead and receptacle connector stack can provide several advantages. For example, the lead insertion force can be reduced as the lead does not have to engage all seals (e.g., wiper seals, proximal seals) of a receptacle connector stack for an entire length of insertion. The lead and seals (e.g., wiper seals) experience less wear as they do not have to engage the entire stroke length during insertion. The power efficiency can be improved as higher power lines can be deployed at larger contact diameters while contacts with lower power specifications can be deployed at smaller contact positions. Larger diameters can create a stiffer, more robust, and more reliable lead. Larger contacts are separated by larger wiper seals which can yield better moisture and contact isolation. Also, stepped smaller contacts facilitates cost reduction. The stepped implantable lead 2200 is further discussed below.

The stepped implantable lead 2200 can include a stepped diameter elongate shaft 2210 with a proximal portion and a distal portion. The proximal portion has a larger diameter than a diameter of the distal portion. In one embodiment, a strength member 2220 (similar to the strength member 1620) or a tapered strength member 2220 can be disposed within the elongate shaft 2210 for improved strength and rigidity. A stem 2230 (similar to the stem 1630) can be coupled to the proximal portion of the stepped diameter elongate shaft 2210.

A plurality of electrical contacts 2211-2216 are disposed on an outer surface of the stepped diameter elongate shaft 2210 and axially spaced from each other, similar to the electrical contacts 1611-1616 or 1811-1816. For example, the plurality of electrical contacts includes a first electrical contact 2211 and a second electrical contact 2212 for power transmission, a third electrical contact 2213 and a fourth electrical contact 2214 for communication, and a fifth electrical contact 2215 and a sixth electrical contact 2216 serving as ground. Each of the electrical contacts 2211-2216 can be coupled to the respective electrical contacts 2211-2216.

In one embodiment, as shown in FIGS. 23 and 24, the stepped diameter elongate shaft 2210 can include a tapered diameter increasing from the distal portion to the proximal portion, where each electrical contact of the six electrical contacts 2211-2216 has a different diameter For example, a first portion can have a first diameter d1 and configured to receive the first contact 2211, a second portion can have a second diameter d2 and configured to receive the second contact 2212, a third portion can have a third diameter d3 and configured to receive the third contact 2213, a fourth portion can have a fourth diameter d4 and configured to receive the fourth contact 2214, a fifth portion can have a fifth diameter d5 and configured to receive the fifth contact 2215, and a sixth portion can have a sixth diameter d6 and configured to receive the sixth contact 2216. Gradual ascending diameter sizes result in a larger contact diameter near the base of the lead, which improves rigidity of the lead. Further, larger sealing surfaces on the base of the lead also promote superior moisture ingress protection, which may allow removal of the vent/septum in the connector stack 2250 as fluid ingress and hydrostatic forces are reduced by having the largest diameter contact 2216 engage last. The stepped diameter configuration can also reduce insertion force and minimize electrical/mechanical contact as the lead is inserted into the receptacle connector stack 2250.

In another embodiment, the stepped diameter elongate shaft 2210 includes three portions (not illustrated) of different diameters, each portion configured to receive two electrical contacts. For example, a first diameter portion can be configured to receive the first and the second electrical contacts for power transmission at the distal portion of the elongate shaft, a second diameter portion can be configured to receive the third and the fourth electrical contacts for communication between the distal portion and the proximal portion of the elongate shaft, and a third diameter portion configured to receive the fifth and the sixth electrical contacts serving as ground at the proximal portion of the elongate shaft. In one embodiment, the second diameter portion (e.g., diameter D3) can have a larger diameter than the first portion (e.g., diameter D1), and the third diameter portion (e.g., diameter D5) has a larger diameter than the second diameter portion (e.g., diameter D3). It can be understood that the present disclosure is not limited to a number of different diameter portions. A person of ordinary skill in the art can modify the stepped diameter elongate shaft 2210 to include two, three, four, five or more stepped portions or a continuous taper design.

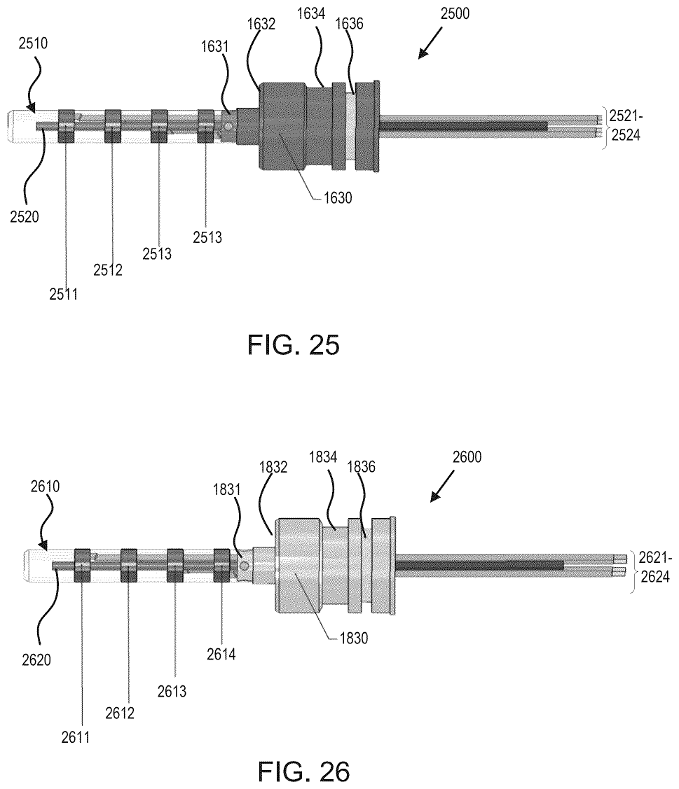

FIGS. 25-26 illustrate implantable leads 2500 and 2600, respectively, with four electrical contacts disposed on an elongate shaft, in accordance with one embodiment. An implantable lead 2500 can be a VAD lead, and another implantable lead 2600 can be a TETS receiver lead, for example. Similar to the implantable leads 1600 and 1800, the leads 2500 and 2600 can include elongate shafts 2510 and 2610, respectively, but with four electrical contacts disposed thereon. In particular, the short, rigid, robust male lead plugs of FIGS. 25-27 stack conductors side by side towards reducing the overall length of the lead while maintaining up to all six electrical lines within a constrained diameter of approximately 3.2 mm.

Employing four electrical contacts facilitate redundancy for one type of electrical contact. For example, the redundancy can be used for any one of power, ground, or communication line related electrical contacts. In one embodiment, the implantable lead 2500 can include a first electrical contact 2511 for serving as ground, a second electrical contact 2512 for transmitting power, and a third electrical contact 2513 and a fourth electrical contact 2514 serving as communication lines. In one embodiment, the other implantable lead 2600 can include a first electrical contact 2611 for transmitting power, a second electrical contact 2612 serving as ground, a third electrical contact 2513 serving as a power line at a fixed voltage (e.g., 5V), and a fourth electrical contact 2614 serving as a communication line.

Furthermore, each of the implantable leads 2500 and 2600 can include four electrical wires 2521-2524 and 2621-2624, respectively. Each electrical wire can be coupled to respective electrical contacts 2511-2514 or 2611-2614. Alternatively, the implantable lead may be configured to include six electrical wires by coupling two electrical wires to one or more of the electrical contacts. For example, two electrical wires can be coupled to each of the first electrical contact and the second electrical contact.

Employing four electrical contacts facilitate reduction in a length of an elongate shaft 2510 and 2610 compared to the implantable leads 1600 or 1800. The reduced length can improve rigidity without incorporating a strength member. Alternatively or in addition, a strength member 2520 or 2620 may be constrained within the elongate shaft 2510 or 2610, respectively, to further improve the rigidity.

FIG. 27 illustrates an implantable lead with three electrical contacts, in accordance with another embodiment. With three electrical contacts, the implantable lead can be shorter than the four contact leads 2500, 2600 or six contact leads 1600, 1800 and rigid enough that a strength member is not required. An implantable lead 2700 can include an elongate shaft 2710 similar to but shorter in length than the elongate shaft 1610 or 1810. The implantable lead 2700 can include a first electrical contact 2711, a second electrical contact 2712, and a third electrical contact 2713 disposed on the elongate shaft 2710. The electrical contacts 2711-2713 can be used for power transmission, communication, and ground. Although only three electrical contacts 2711-2713 are used, the implantable lead 2700 can include six electrical wires disposed within the elongate shaft 2710 and coupled to the electrical contacts 2711-2713, as shown. For example, each electrical contact (e.g., the first contact 2711) can be coupled with two electrical wires (e.g., wires 271 and 2722). In one embodiment, each electrical contact can be split into two half-moon shapes and isolated from each other to create full redundancy along a radial direction. Accordingly, each half of the electrical contact can be coupled to one electrical wire.

In one or more embodiments of the present disclosure, an implantable medical device comprising an implanted connector with booster sealing elements for improved sealing performance comprises a housing, a header coupled to the housing, and at least one receptacle connector stack disposed in the header. The at least one receptacle connector stack includes a plurality of electrical contacts and a plurality of wiper seals. Each electrical contact of the plurality of electrical contacts is separated by a corresponding wiper seal. A first sealing element is disposed at a proximal end of the at least one receptacle connector stack and a second sealing element is disposed at a distal end of the at least one receptacle connector stack. Optionally, the first sealing element and the second sealing element may comprise O-rings. Optionally, a diameter of each O-ring is within a range of 0.115″ to 0.375″ for the IDs and 0.145″ to 0.515″ for the ODs. Optionally, the first sealing element is configured to inhibit fluid ingress and stray electrical currents at the proximal end of the at least one receptacle connector stack from an outside environment, and the second sealing element is configured to inhibit fluid ingress and stray electrical currents at the distal end of the at least one receptacle connector stack from the outside environment. Optionally, the implantable medical device further includes a proximal seal housing configured to receive the first sealing element. Likewise, a distal seal housing is configured to receive the second sealing element. Optionally, the proximal seal housing and the distal seal housing are separate components assembled with the at least one receptacle connector stack. Optionally, the proximal seal housing and the distal seal housing are integrally formed with the at least one receptacle connector stack. Optionally, the proximal seal housing includes a retention mechanism configured to engage with the at least one receptacle connector stack and the header. The retention mechanism includes at least one of a press fit, a crush rib fit, a snap fit, a retaining ring, or a threaded fit. Optionally, the implantable medical device further includes a third sealing element disposed adjacent to the first sealing element and further proximal of the at least one receptacle connector stack to restrict a bounce back effect caused by one or more of the plurality of wiper seals. Optionally, the implantable medical device is a ventricular assist device (VAD) implantable controller configured to generate control signals to control a blood flow and provide power to the VAD. Optionally, the header includes two receptacle connectors stacks spaced apart from each other, each connector stack including six contacts for power and communication with the VAD and TETS. Optionally, the at least one receptacle connector stack includes a first receptacle connector stack spaced from and electrically isolated from a second receptacle connector stack.

In one or more embodiments of the present disclosure, a fully implantable left ventricle assist system (FILVAS) includes a heart pump, an implantable transcutaneous energy transmission system (TETS) receiver, a first implantable lead, a second implantable lead, and an implantable controller. The heart pump is configured for pumping blood from a ventricle of a heart of a patient to an artery to supplement or replace pumping of blood by the ventricle to the artery. The TETS receiver is configured for receiving and transmitting power to continuously operate the heart pump. The implantable controller is communicably coupled to the heart pump via the first implantable lead and to the TETS receiver via the second implantable lead. Optionally, the implantable controller includes a housing, a header coupled to the housing, a first receptacle connector stack disposed in the header and configured to receive the first implantable lead and establish an electrical coupling between the implantable controller and the heart pump, and a second receptacle connector stack disposed in the header and configured to receive the second implantable lead and establish an electrical coupling between the implantable controller and the TETS receiver. Each of the first receptacle connector stack and the second receptacle connector stack includes a plurality of electrical contacts configured to couple with the respective implantable lead, a plurality of wiper seals, each electrical contact being isolated by a corresponding wiper seal, a proximal sealing element disposed at a proximal end of the respective receptacle connector stack, and a distal sealing element disposed at a distal end of the respective receptacle connector stack. Optionally, the proximal sealing elements and the distal sealing elements are O-rings. Optionally, the fully implantable left ventricle assist system further includes, for each of the first receptacle connector stack and the second receptacle connector stack, a proximal seal housing configured to receive the proximal sealing element and coupleable to the proximal end of the respective receptacle connector stack. A distal seal housing is configured to receive the distal sealing element and is coupleable to the distal end of the respective receptacle connector stack. Optionally, each proximal seal housing includes a retention mechanism configured to engage with the respective receptacle connector stack and the header. Optionally, the header further includes a bounce back reducer disposed within the header and located proximal of the respective proximal sealing element. The bounce back reducer is configured to engage the respective implantable lead and restrict a bounce back effect experienced by the respective implantable lead that are caused by forces exerted by one or more of the plurality of wiper seals upon insertion of the respective implantable lead into the respective receptacle connector stack. Optionally, the bounce back reducer is a canted spring, synching feature, an O-ring, or a complaint member. Optionally, the header further includes a cassette with a groove to receive the bounce back reducer. Optionally, the header further includes a vent or a septum located distal to the distal sealing element of the respective receptacle connector stack to prevent hydrostatic locking between the respective implantable lead and the respective receptacle connector stack. The distal sealing element inhibits ingress of fluid from the vent or the septum. Optionally, the first implantable lead includes electrical contacts configured to couple with a corresponding electrical contacts of the first receptacle connector stack, and an electrical cable to facilitate communication and power between the implantable controller and the heart pump. The second implantable lead comprises electrical contacts configured to couple with a corresponding electrical contact of the second receptacle connector stack, and an electrical cable to facilitate communication and power between the implantable controller and the TETS receiver. Optionally, the first receptacle connector stack is spaced from and electrically isolated from the second receptacle connector stack by the respective proximal sealing elements and the respective distal sealing elements.

In one or more embodiments of the present disclosure, an implantable lead of an implantable medical device is described. The implantable lead includes an elongate shaft, a stem, a plurality of electrical contacts, a strength member, and a plurality of wires. The elongate shaft has a proximal end and a distal end, the elongate shaft having a diameter of approximately 3.2 mm, a stem coupled to the proximal end of the elongate shaft. The plurality of electrical contacts are disposed on an outer surface of the elongate shaft and axially spaced from each other. The plurality of electrical contacts include first and second electrical contacts for power transmission, third and fourth electrical contacts for communication, and fifth and sixth electrical contacts serving as ground. The strength member is disposed axially within the elongate shaft. The plurality of wires are electrically coupled to the plurality of electrical contacts and extending axially within the elongate shaft. The plurality of wires include a first wire and a second wire electrically coupled to the first and the second electrical contacts, respectively; a third wire and a fourth wire electrically coupled to the third and the fourth electrical contacts, respectively; and a fifth wire and a sixth wire electrically coupled to the fifth and the sixth electrical contacts, respectively. The strength member and the plurality of wires are electrically isolated from each other are configured to be constrained within the diameter of the elongate shaft. Optionally, the plurality of wires are radially disposed approximately 60 degree from each other and circumferentially around the strength member. Optionally, each of the plurality of electrical contacts comprises a ring contact, wherein the ring contacts are coaxial and linearly spaced from each other along the elongate shaft. Optionally, the electrical contacts and the electrical wires are made of corrosion resistant and electrically conductive material. Optionally, the implantable lead further includes an insulation cover disposed over each wire of the plurality of wires. Optionally, the plurality of electrical contacts are separated by a molded insulative material for electrical isolation. Optionally, the first and the second electrical contacts for power transmission are disposed at a distal portion of the elongate shaft. The fifth and the sixth electrical contacts serving as ground are disposed at a proximal portion of the elongate shaft. The third and the fourth electrical contacts for communication are disposed between the proximal portion and the distal portion of the elongate shaft. Optionally, the stem includes a shoulder at a distal portion of the stem where the elongate shaft is configured to receive a sealing element of a header assembly, and a groove configured to receive a locking assembly for securely coupling the lead to the header assembly of a second implantable medical device. Optionally, the implantable lead comprises a ventricular assist device (VAD) lead or a transcutaneous energy transmission system (TETS) receiver lead. Optionally, the implantable lead is coupled to a receptacle connector stack of an implantable controller.

In one or more embodiments of the present disclosure, a fully implantable left ventricle assist system (FILVAS) is described. The system includes an implantable heart pump, an implantable transcutaneous energy transmission system (TETS) receiver, a first implantable lead, a second implantable lead, and an implantable controller. The implantable heart pump configured for pumping blood from a ventricle of a heart of a patient to an artery to supplement or replace pumping of blood by the ventricle to the artery. The implantable transcutaneous energy transmission system (TETS) receiver configured for receiving and transmitting power to continuously operate the implantable heart pump. The first implantable lead coupled to the implantable heart pump. The first lead includes a first elongate shaft, a first set of six electrical contacts coaxially mounted on an outer surface of the first elongate shaft, a first set of six wires disposed within the first elongate shaft, each wire electrically coupled to a respective electrical contact of the first set of six electrical contacts at one end and to the implantable heart pump at an opposite end; and a first strength member disposed within the first elongate shaft and electrically isolated from the first set of six wires. The second implantable lead coupled to the TETS receiver. The second lead includes a second elongate shaft, a second set of six electrical contacts coaxially mounted on an outer surface of the second elongate shaft, a second set of six wires disposed within the second elongate shaft, each wire electrically coupled to a respective electrical contact of the second set of six electrical contacts at one end and the TETS receiver at an opposite end; and a second strength member disposed within the second elongate shaft and electrically isolated from the second set of six wires. The second implantable lead is larger in diameter than the first implantable lead. The implantable controller includes a first receptacle connector stack configured to receive the first implantable lead to electrically couple the implantable heart pump to the implantable controller, and a second receptacle connector stack configured to receive the second implantable lead to electrically couple the TETS receiver to the implantable controller. The first receptacle connector stack spaced apart from the second receptacle connector stack. Optionally, the first set of six electrical contacts of the first implantable lead comprises a first and a second electrical contacts for power transmission, a third and a fourth electrical contacts for communication, and a fifth and a sixth electrical contacts for serving as ground. The second set of six electrical contacts of the second implantable lead comprises a first and a second electrical contacts for power transmission, a third and a fourth electrical contacts for communication, and a fifth and a sixth electrical contacts for serving as ground. Optionally, the first implantable lead includes the first and the second electrical contacts for power transmission are disposed at a distal portion of the first implantable lead. The fifth and the sixth electrical contacts serving as ground are disposed at a proximal portion of the first implantable lead. The third and the fourth electrical contacts for communication are disposed between the proximal portion and the distal portion of the first implantable lead. Optionally, the second implantable lead includes a first electrical contact serving as a first volt supply disposed at a distal portion of the second implantable lead, a second electrical contact serving as a ground disposed adjacent and proximal the first electrical contact, a third electrical contact serving as a power terminal disposed adjacent and proximal the second electrical contact, a fourth electrical contact serving as a communication line disposed adjacent and proximal the third electrical contact, a fifth electrical contact serving as another ground disposed adjacent to and proximal the fourth electrical contact, and a sixth electrical contact serving as another power terminal disposed at a proximal portion of the second implantable lead. Optionally, a proximal portion of the second implantable lead is larger in diameter than a proximal portion of the first implantable lead. Optionally, the first implantable lead includes a first stem disposed at a proximal portion of the first elongate shaft and the second implantable lead includes a second stem disposed at a proximal portion of the second elongate shaft. A diameter of the second stem is larger than a diameter of the first stem. Optionally, each of the first stem and the second stem includes a shoulder for mounting a seal and a groove for locking the respective lead to a header assembly of the implantable controller. Optionally, the first receptacle connector stack and the second receptacle connector stack each include a proximal sealing element and a distal sealing element. Optionally, each of the first implantable lead and the second implantable lead do not require tightening a set screw to electrically activate one or more lines of conductions.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

While certain embodiments have been described, these embodiments have been presented by way of example only, and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

All references, including publications, patent applications, and patents, cited herein are hereby incorporated by reference to the same extent as if each reference were individually and specifically indicated to be incorporated by reference and were set forth in its entirety herein.

1. An implantable medical device comprising:
a housing;
a header coupled to the housing; and
at least one receptacle connector stack disposed in the header, the at least one receptacle connector stack comprising a plurality of electrical contacts and a plurality of wiper seals;
wherein each electrical contact of the plurality of electrical contacts is separated by a corresponding wiper seal, and wherein a first sealing element is disposed at a proximal end of the at least one receptacle connector stack and a second sealing element is disposed at a distal end of the at least one receptacle connector stack.

2. The implantable medical device of claim 1, wherein the first sealing element and the second sealing element comprise O-rings.

3. The implantable medical device of claim 2, wherein a diameter of each O-rings is within a range of 0.115 to 0.375 inches for the inner diameters and 0.145 to 0.515 inches for the outside diameters.

4. The implantable medical device of claim 1, wherein:
the first sealing element is configured to inhibit fluid ingress and stray electrical currents at the proximal end of the at least one receptacle connector stack from an outside environment, and
the second sealing element is configured to inhibit fluid ingress and stray electrical currents at the distal end of the at least one receptacle connector stack from the outside environment.

5. The implantable medical device of claim 1, further comprising:
a proximal seal housing configured to receive the first sealing element; and
a distal seal housing configured to receive the second sealing element.

6. The implantable medical device of claim 5, wherein the proximal seal housing and the distal seal housing are integrally formed within the at least one receptacle connector stack.

7. The implantable medical device of claim 5, wherein the proximal seal housing comprises a retention mechanism configured to engage with the at least one receptacle connector stack and the header, wherein the retention mechanism comprises at least one of:
a press fit, wherein an outer periphery of the proximal seal housing is sized to tightly fit with an inner periphery of the header at a proximal end;
a crush rib fit, wherein the outer periphery of the proximal seal housing includes one or more ribs configured to dig into the inner periphery of the header at the proximal end;
a snap fit, wherein the outer periphery of the proximal seal housing includes one or more snapping elements configured to engage with the inner periphery of the header at the proximal end;
a retaining ring attached on the outer periphery of the proximal seal housing to hold the proximal housing in place against the housing and restrict axial movement at the proximal end; or
a threaded fit, wherein the outer periphery of the proximal seal housing includes threads configured to engage with threads on the inner periphery of the housing at the proximal end.

8. (canceled)

9. The implantable medical device of claim 1, further comprising:
a third sealing element disposed adjacent to the first sealing element and further proximal of the at least one receptacle connector stack to restrict a bounce back effect caused by one or more of the plurality of wiper seals.

10. The implantable medical device of claim 1, wherein the implantable medical device is a ventricular assist device (VAD) implantable controller configured to generate control signals to control a blood flow and provide power to the VAD.

11. The implantable medical device of claim 10, wherein the header comprises two receptacle connectors stacks spaced apart from each other, each connector stack including six contacts for power and communication with the VAD and an implantable transcutaneous energy transmission system (TETS) receiver.

12. The implantable medical device claim 1, wherein the at least one receptacle connector stack comprises a first receptacle connector stack spaced from and electrically isolated from a second receptacle connector stack.

13. A fully implantable left ventricle assist system comprising:
a heart pump configured for pumping blood from a ventricle of a heart of a patient to an artery to supplement or replace pumping of blood by the ventricle to the artery;
an implantable transcutaneous energy transmission system (TETS) receiver configured for receiving and transmitting power to continuously operate the heart pump;
a first implantable lead;
a second implantable lead; and
an implantable controller communicably coupled to the heart pump via the first implantable lead and to the TETS receiver via the second implantable lead,
wherein the implantable controller comprises:
a housing;
a header coupled to the housing;
a first receptacle connector stack disposed in the header and configured to receive the first implantable lead and establish an electrical coupling between the implantable controller and the heart pump; and
a second receptacle connector stack disposed in the header and configured to receive the second implantable lead and establish an electrical coupling between the implantable controller and the TETS receiver, and
wherein each of the first receptacle connector stack and the second receptacle connector stack comprise: a plurality of electrical contacts configured to couple with the respective implantable lead; a plurality of wiper seals, each electrical contact being isolated by a corresponding wiper seal; a proximal sealing element disposed at a proximal end of the respective receptacle connector stack;
and a distal sealing element disposed at a distal end of the respective receptacle connector stack.

14. The fully implantable left ventricle assist system of claim 13, wherein the proximal sealing elements and the distal sealing elements are O-rings.

15. The fully implantable left ventricle assist system of claim 13, further comprising for each of the first receptacle connector stack and the second receptacle connector stack:
a proximal seal housing configured to receive the proximal sealing element and coupleable to the proximal end of the respective receptacle connector stack, wherein each proximal seal housing comprises a retention mechanism configured to engage with the respective receptacle connector stack and the header; and
a distal seal housing configured to receive the distal sealing element and coupleable to the distal end of the respective receptacle connector stack.

16. (canceled)

17. The fully implantable left ventricle assist system of claim 13, wherein the header further comprises:
a bounce back reducer disposed within the header and located proximal of the respective proximal sealing element, wherein the bounce back reducer is configured to engage the respective implantable lead and restrict a bounce back effect experienced by the respective implantable lead that are caused by forces exerted by one or more of the plurality of wiper seals upon insertion of the respective implantable lead into the respective receptacle connector stack.

18. The fully implantable left ventricle assist system of claim 17, wherein the bounce back reducer is a canted spring, an O-ring, or a complaint member.

19. The fully implantable left ventricle assist system of claim 17, wherein the header further comprises a cassette with a groove to receive the bounce back reducer.

20. The fully implantable left ventricle assist system claim 13, wherein the header further comprises:
a vent located distal to the distal sealing element of the respective receptacle connector stack to prevent hydrostatic locking between the respective implantable lead and the respective receptacle connector stack,
wherein the distal sealing element inhibits ingress of fluid from the vent.

21. The fully implantable left ventricle assist system of claim 13, wherein:
the first implantable lead comprises electrical contacts configured to couple with a corresponding electrical contacts of the first receptacle connector stack, and an electrical cable to facilitate communication and power between the implantable controller and the heart pump; and
the second implantable lead comprises electrical contacts configured to couple with a corresponding electrical contact of the second receptacle connector stack, and an electrical cable to facilitate communication and power between the implantable controller and the TETS receiver.

22. The fully implantable left ventricle assist system of claim 13, wherein the first receptacle connector stack is spaced from and electrically isolated from the second receptacle connector stack by the respective proximal sealing elements and the respective distal sealing elements.

23.-48. (canceled)
